(19) United States
(12) Patent Application Publication
(10) Pub. No.: US 2008/0182309 A1
(43) Pub. Date: Jul. 31, 2008

(54) METHOD AND APPARATUS FOR MAGNETIC FERMENTATION

(75) Inventor: VLADIMIR VLAD, AMES, IA (US)

Correspondence Address: MCKEE, VOORHEES & SEASE, P.L.C., 801 GRAND AVENUE, SUITE 3200, DES MOINES, IA, 50309-2721, US

(73) Assignee: EMTECH, LLC, Johnston, IA (US)

(21) Appl. No.: 11/842,743

(22) Filed: Aug. 21, 2007

Related U.S. Application Data
(60) Provisional application No. 60/823,023, filed on Aug. 21, 2006.

(51) Int. Cl. C12N 13/00; C12P 7/06; C12M 1/42; H01F 7/08

(52) U.S. Cl. 435/161; 435/173.1; 435/283.1; 435/287.1; 335/219

(57) ABSTRACT

A method for magnetic fermentation is provided which includes subjecting a biological material in a medium to a static magnetic field in order to affect fermentation of the biological material into a fermented product. The fermentation reaction may occur in an acidic or alkaline medium. The magnetic field may be a positive or negative magnetic field. The magnetic field or other parameters associated with the fermentation process may be monitored with one or more sensors and the magnetic field may be modulated accordingly.

### CROSS-REFERENCE TO RELATED APPLICATIONS

This application claims priority under 35 U.S.C. § 119 to provisional application Ser. No. 60/823,023 filed Aug. 21, 2006, herein incorporated by reference in its entirety.

### BACKGROUND OF THE INVENTION

The present invention relates to improved fermentation. Fermenters or bioreactors are used, for example, to optimize growth conditions of various strains of bacteria and tissue culture cells. Fermentation is used in numerous applications, including production of fuel alcohols such as ethanol, production of distilled beverages, food manufacturing, textiles, and pharmaceuticals. Despite the widespread use and availability of fermenters, it is still desired to be able to improve fermentation processes such as by increasing fermentation rates and yields and exercise more control over fermentation processes.

### BRIEF SUMMARY OF THE INVENTION

Therefore, it is a primary object, feature, or advantage of the present invention to improve over the state of the art.

It is a further object, feature, or advantage of the present invention to provide an improved fermentation process.

A still further object, feature, or advantage of the present invention is to improve the ability to control a fermentation process while maintaining a chemical free environment.

A further object, feature, or advantage of the present invention is to provide a fermenter which is self-regulating in order to optimize the fermentation process.

Another object, feature, or advantage of the present invention is to provide a biofermenter capable of providing improved ethanol production from biological material including plant material such as corn.

Another object, feature, or advantage of the present invention is to ferment carbohydrates which may be used for food or in the production of ethanol.

One or more of these and/or other objects, features, or advantages of the present invention will become apparent from the specification and claims that follow.

According to one aspect of the present invention, a method for magnetic fermentation is provided which includes subjecting a biological material in a medium to a magnetic field in order to affect fermentation of the biological material into a fermented product. The fermentation reaction may occur in an acidic or alkaline medium. The magnetic field may be a positive or negative magnetic field. The magnetic field or other parameters associated with the fermentation process may be monitored with one or more sensors and the magnetic field may be modulated accordingly.

According to another aspect of the present invention, a method for producing ethanol includes subjecting to a positive magnetic field of about 2000 to about 3000 Gauss a medium comprising a biological material and a microorganism or an enzymatic preparation or a combination thereof, wherein the microorganism or the enzymatic preparation is capable of fermenting the biological material to produce ethanol. The biological material may be any of a number of different types of fermentable materials.

According to another aspect of the present invention, a method for magnetic fermentation includes controlling a fermentation process by subjecting a biological material in a medium to a DC magnetic field having a magnitude of between 2000 to 3000 Gauss and electronically monitoring the fermentation process to generate fermentation data. The step of controlling comprises adjusting the magnitude of the DC magnetic field.

According to another aspect of the present invention a magnetic fermentation system includes a fermentation vessel for containing biological material and a medium, a magnetic field component for applying a magnetic field to the medium during a fermentation process within the fermentation vessel, wherein the magnetic field component is configured to create a magnetic field having a magnitude of between 2000 to 3000 Gauss. The magnetic fermentation system may include an intelligent control electrically connected to the magnetic field component and adapted for controlling the magnetic field applied by the magnetic field component. The magnetic fermentation system may be configured for applying the magnetic field to medium within staging operatively connected to the vessel. The staging may include a recycle stage.

According to another aspect of the present invention, a magnetic field generating device for applying a magnetic field to a fluid flowing through a conduit is provided. The device includes a plurality of magnetic modules, each of the magnetic modules being substantially U-shaped for positioning on the conduit, each of the magnetic modules having a base and legs extending from the base with an electromagnet at the base and permanent magnets near the legs. The plurality of magnetic modules being arranged in alternating fashion such that the base of each magnetic module is opposite the base of any immediately adjacent magnetic module.

### BRIEF DESCRIPTION OF THE DRAWINGS

FIG. 1 is a block diagram of one embodiment of the present invention.

FIG. 2 is a flow diagram illustrating one embodiment of the methodology of the present invention.

FIG. 3 is a graph illustrating magnetic field versus distance.

FIG. 4 is a graph illustrating magnetic field versus distance between various points associated with a magnetic fermenter according to one embodiment of the present invention.

FIG. 5 is a graph illustrating various test runs of a magnetic fermenter according to one embodiment of the present invention.

FIG. 6 is a perspective view of one embodiment of a magnetic fermenter according to the present invention.

FIG. 7 is a perspective view of one embodiment of magnetic agitators used in the magnetic fermenter of the present invention and a gauss meter.

FIG. 8 is partial perspective view showing one embodiment of the magnetic fermenter of the present invention.

FIG. 9 is a view illustrating the electromagnetic fields associated with one embodiment of a magnetic fermenter of the present invention.

FIG. 10 illustrates one embodiment of a magnetic fermenter where permanent magnets are used which are positioned within the tank or vessel.

FIG. 11 illustrates the magnetic fermenter of FIG. 10 along with field lines.

FIG. 12 is a perspective view of one embodiment of a device for applying a magnetic field.

FIG. 13 is a top view of the device of FIG. 12.

FIG. 14 is a front view of the device of FIG. 12.

FIG. 15 is a top view of a magnetic module.

FIG. 16 is a side view of the magnetic module of FIG. 15.

FIG. 17 is a sectional view of the magnetic module.

FIG. 18 is an electrical schematic for controlling the device of FIG. 12.

FIG. 19 is a block diagram showing one embodiment of the present invention where a solenoid device is used to generate an electromagnetic field applied to fluid circulating external to a fermentation tank.

FIG. 20 is a diagram showing the solenoid device of FIG. 19.

FIG. 21 is a pictorial representation of magnetic fields associated with the solenoid device.

FIG. 22 is one embodiment of a method of producing ethanol using magnetic fermentation.

FIG. 23 is a graph providing an ethanol production comparison.

FIG. 24 illustrates glucose consumption and ethanol production curves, for a 5 liter fermentation system using HPLC analysis.

FIG. 25 illustrates growth curves for a 5 liter fermentation vessel.

FIG. 26 illustrates glucose consumption and ethanol production curves for a 100 liter fermentation system using HPLC analysis.

FIG. 27 is a graph comparing results of 5-L to 100-L runs.

FIG. 28 is a graph comparing the results of glucose consumption and ethanol production for 100 L and 5 L fermentation systems.

FIG. 29 is a bar graph comparing maximum biomass production rate achieved using fermentation methods with and without magnetism.

FIG. 30 is a bar graph comparing the maximum consumption of glucose and production rates of ethanol achieved using fermentation methods with and without magnetism.

### DETAILED DESCRIPTION OF THE PREFERRED EMBODIMENT

The present invention provides for method and devices which may be used for applying and/or controlling magnetic fields during fermentation processes. Without wishing to be bound by this theory, it is contemplated that the use of a magnetic field and the ability to modulate the magnetic field during fermentation confers several advantages. These include but are not limited to any of the following: speeding up the dissolving of oxygen in the medium/water, increasing the rate of enzyme or bacteria's cell division, and disrupting the water's hydrogen bonding to generate unstructured water with fewer hydrogen bonds to provide a more reactive environment. Importantly, cells convert a larger faction of the sugar substrate towards cell mass production as the amount of oxygen available to the cells speeds up which allows the fermentation rate to increase.

The methods of the present invention may be used in producing any number of products whose production employs fermentation. Examples include without limitation: cellulosic ethanol that uses hydrolysis of cellulose followed by fermentation of the generated free sugars; ethanol produced by methods such as the simultaneous saccharification and fermentation of a biological material such as glucose or inulin (Ohta et al. Production of high concentrations of ethanol from inulin by simultaneous saccharification and fermentation using Aspergillus niger and Saccharomyces cerevisiae. Appl Environ Microbiol. 1993 March; 59(3):729-33); polymeric hexose and pentose sugars in cellulose and hemicellulose; glucose, lactic acid produced by the fermentation of sugars and the like. Accordingly, methods of the present invention may be used in the fermentation of a biological material to ethanol, the Simultaneous Saccharification and Fermentation (SSF) of a biological material to ethanol, and fermentation of a biological material to lactic acid.

The present invention provides for applying a magnetic field to affect a fermentation process. To assist in describing the invention, the basic process of applying magnetic fields to fermentation is described. Next, various embodiments for producing the magnetic field are described. Finally, examples of the process are provided and results from various fermentation processes are given. Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the invention.

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Applying Magnetic Fields

A magnetic field is created and applied in order to affect a fermentation process. Different types of magnetic fields are used in different embodiments. The magnetic fields may be created by permanent magnets or electromagnets, although variable DC controlled electromagnets provide for convenient adjustment of magnetic fields. Generally, the magnetic fields applied are monopole or substantially monopole. The monopole magnetic fields may be positive or negative, depending upon the effect desired. The magnetic fields are controlled in a manner that assists the fermentation process, by increasing the rate of enzyme or bacteria's cell division. When the magnetic field removes positively charged calcium ions that help bind them together, this is loosening the membrane structure and is increasing its permeability, resulting in extra free calcium leaking into the cell from outside (normally there is about a thousand times greater concentration of calcium on the outside than on the inside). This process stimulate metabolism and cell multiplication (cells normally regulate their rate of metabolism by controlling their internal calcium concentration). By “chemical free environment” it is understood that applying a magnetic field does not involve use of any chemicals. A feedback loop can be established so that the intensity of the magnetic field can be varied as data regarding the fermentation process is monitored. A submersible Gauss meter or other type of magnetic field sensor can be placed in the fermenter or otherwise used to measure the magnetic field being applied. In addition oxidation reduction potential (ORP) and pH can be monitored. The magnetic fermenter essentially controls the pH levels through the amplifying process of the magnetic field. The magnetic fields can be created by generating magnetic fields within a fermentation vessel, outside of the fermentation vessel with the magnetic field directed inwardly, or during staging operations that occur outside of the fermentation vessel, such as applying the magnetic field to a pipe associated with a recycle stage.

All fermentation data received from the fermenter's computer system or other intelligent control system may be monitored and used to adjust the application of the magnetic fields. For example, where a DC power supply is used, the fermentation data may be conveyed as data correction for the electromagnets DC Power Supply. A bridge software program may interpret the data and modulate the magnetic field created in order to affect the fermentation process. In this way, the fermenter becomes self-regulating; the magnetic field for enzymatic stimulation, the pH (by increasing the voltage or by reversing the electric field polarity to the magnets) may be regulated based on a production curve and the magnetic field can be modulated such as to maintain a desired pH.

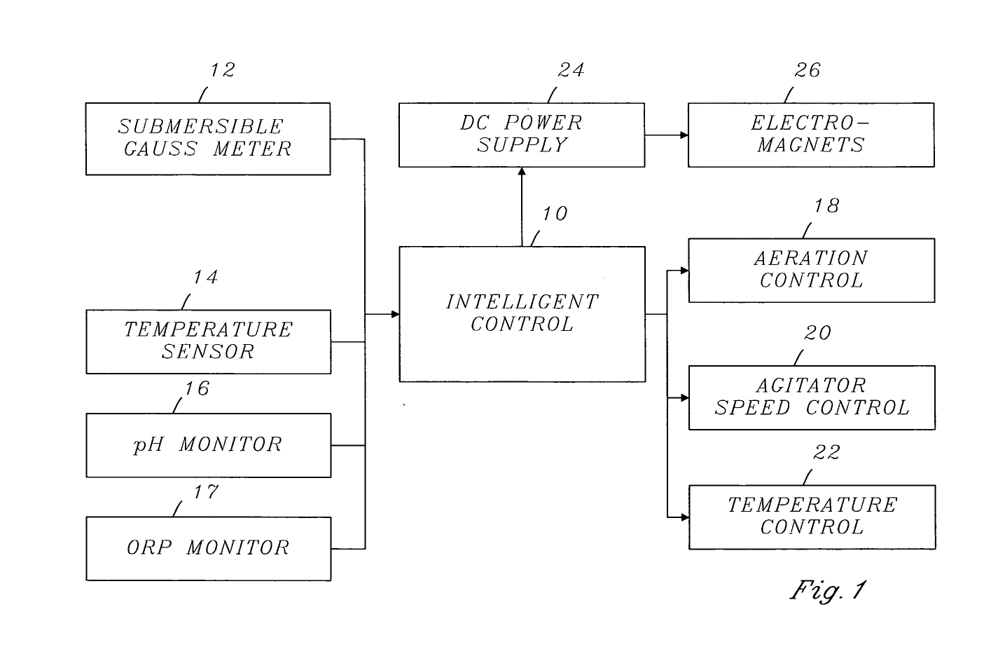

FIG. 1 is a block diagram of one embodiment of a control system for a magnetic fermentation system of the present invention. As shown in FIG. 1 an intelligent control 10 is provided. The intelligent control 10 may be implemented in hardware or software on a computer, processor, microcontroller, integrated circuit, or other type of intelligent control. The intelligent control 10 is electrically connected to one or more types of sensors. Shown in FIG. 1 are a submersible gauss meter 12, a temperature sensor 14, a pH monitor 16, and an oxidation reduction potential (ORP) monitor 17. In addition, the intelligent control 10 is electrically connected to one or more types of controls such as an aeration control 18, an agitator speed control 20, and a temperature control 22. Although various types of sensors and controls are shown and described herein the present invention contemplates that not all of these types of sensors and controls need be used. Further, additional types of sensors and controls may be used as determined to be most appropriate in a particular fermentation process. Also connected to the intelligent control 10 is a DC power supply 24. The DC power supply 24 is electrically connected to a plurality of electromagnets 26.

As shown in FIG. 1, fermentation data from the sensors shown or other types of sensors associated with fermentation data is received by the intelligent control 10. Based on analysis of the information received, the intelligent control 10 can appropriately control any of the controls as well as the DC power supply 24. By properly adjusting the DC power supply 24, the intelligent control 10 effects an appropriate change in the induced magnetic field and therefore the fermentation process. Thus, the fermentation system may be self-regulating or self-adjusting in order to optimize the fermentation process to reach a desired goal such as fermentation in the least amount of time, a particular fermentation rate, or a particular yield. For example, the magnetic field can be increased or decreased to control the magnetic field for enzymatic stimulation. The pH can be modified by increasing the voltage or reversing the electric field polarity to the magnets. One way of regulating the fermentation is to base the regulation on a production curve associated with the fermentation process. Thus, for example, in ethanol production, the fermentation process may be based on an ethanol production curve.

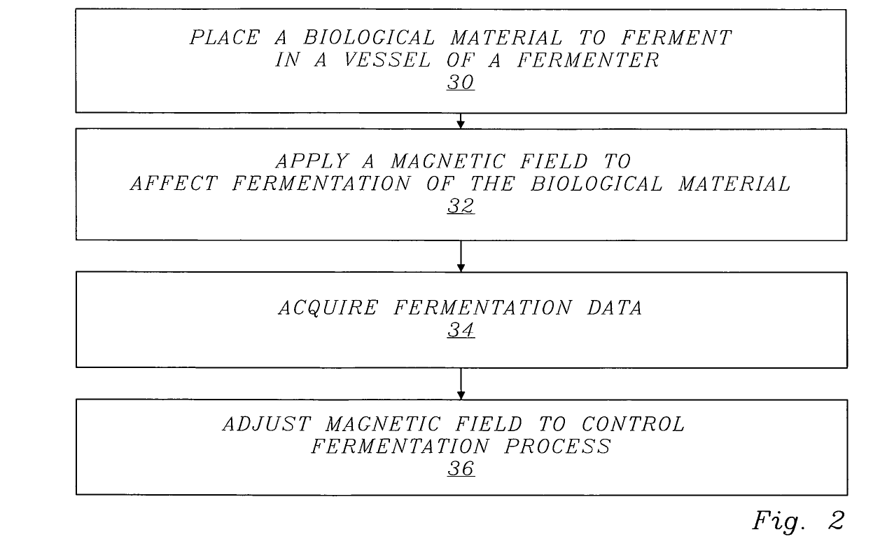

FIG. 2 is a flow diagram illustrating one embodiment of a method of the present invention. As shown in FIG. 2, in step 30, a biological material to be fermented is placed within a vessel or tank of a fermenter. Next, in step 32, a magnetic field is applied throughout the vessel in order to affect fermentation of the biological material. In step 34, fermentation data is acquired through the use of sensors or otherwise. Examples of such sensors include, but are not limited to temperature sensors, pH sensors, chemical sensors, and other types of sensors. The fermentation data acquired may also be human observable data regarding the fermentation process. In step 36, the magnetic field is adjusted in order to control the fermentation process.

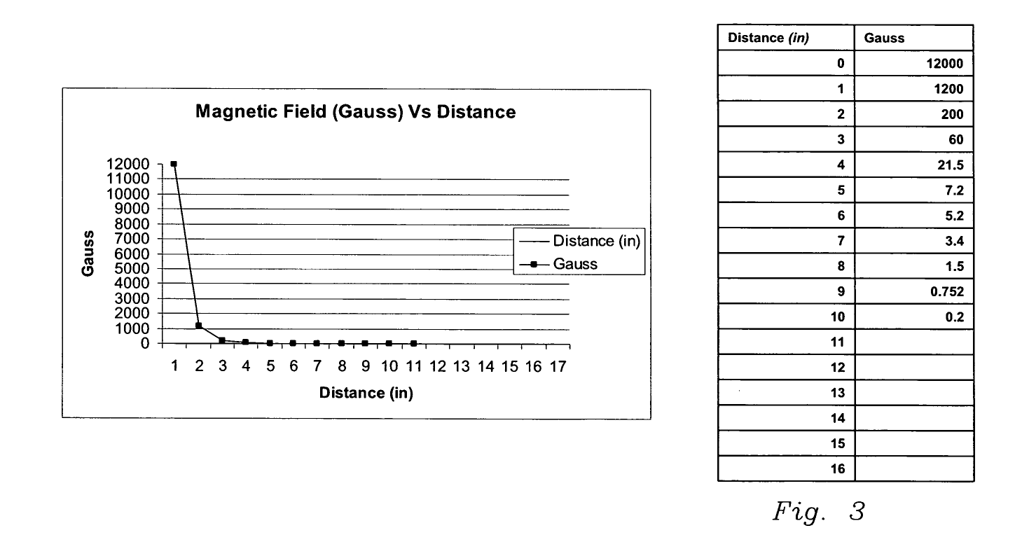

FIG. 3 is a graph illustrating magnetic field versus distance. Note that magnetic field as measured in Gauss varies inversely with the square of the distance. The present invention allows for placing magnets around the vessel of the fermenter to thereby provide a more uniform and consistent magnetic field.

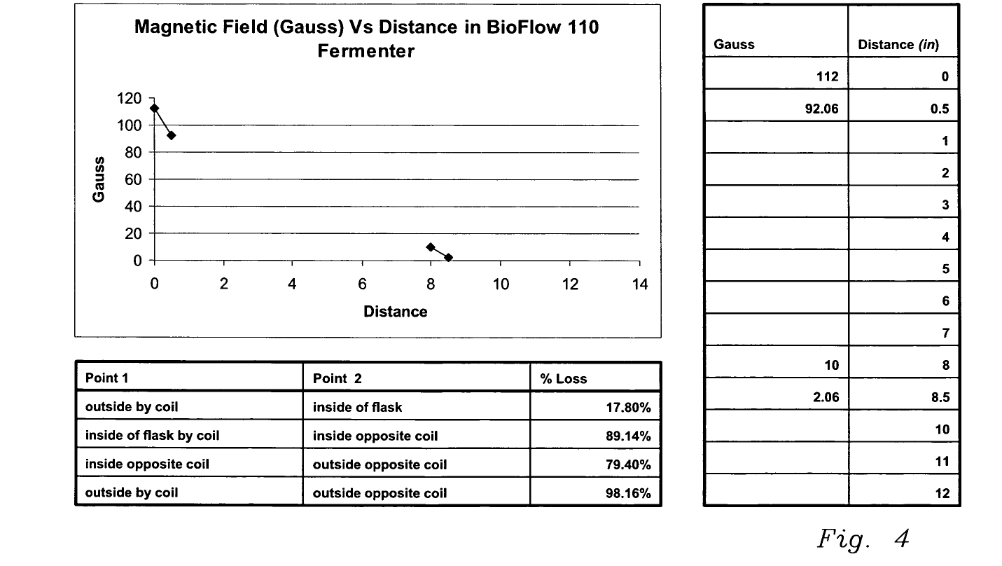

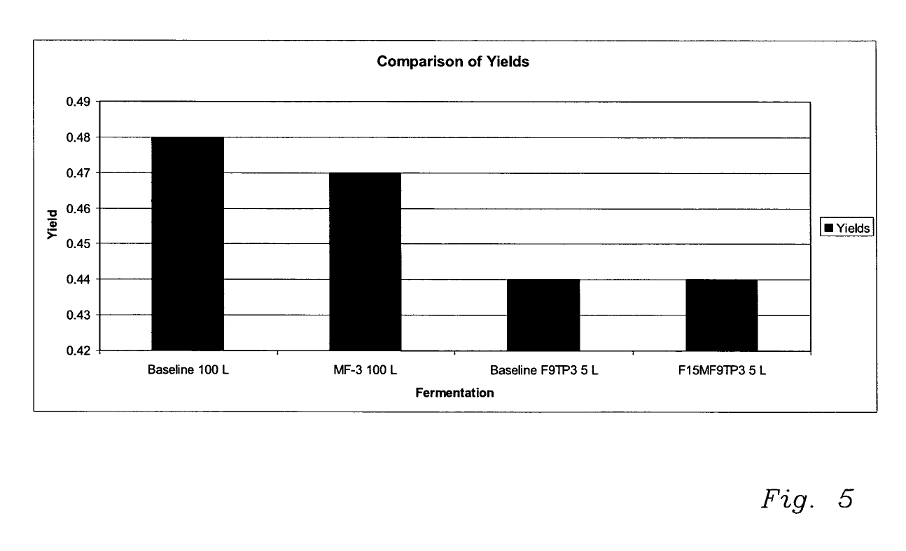

FIG. 4 is a graph illustrating magnetic field versus distance between various points associated with a fermenter. The magnetic field loss was determined between a first point outside of the vessel by a coil and a second point inside of the vessel. The magnetic field loss was also determined between a first point inside of the vessel by the coil and a second point inside the opposite coil. The magnetic field loss was also determined between a first point inside the opposite coil and a second point outside the opposite coil. The magnetic field loss was also determined between a first point outside of the coil and a second point outside of the opposite coil. Where the magnetic field is generated on the outside of the fermentation vessel, the magnetic field loss is preferably minimized to provide as uniform of a magnetic field as possible.

Where magnetic fermentation is applied using a monopole positive field, it has been observed that dissolved oxygen (D) increases by about 26 percent in the first 30 minutes. It has further been observed that a positive and substantially monopole magnetic field increases the enzymes' metabolic activity by about 25 percent. Thus, the application of the positive field provides significant benefits and advantages.

### II. Producing Magnetic Fields

Various embodiments of devices for producing magnetic fields may be used. These include embodiments where permanent magnets are used, where electromagnets are used, where the magnetic field is applied from outside of a vessel inwardly, where the magnetic field is applied from within the vessel, and where the magnetic field is applied in a staging such as the recycle stage, as opposed to within the fermentation vessel.

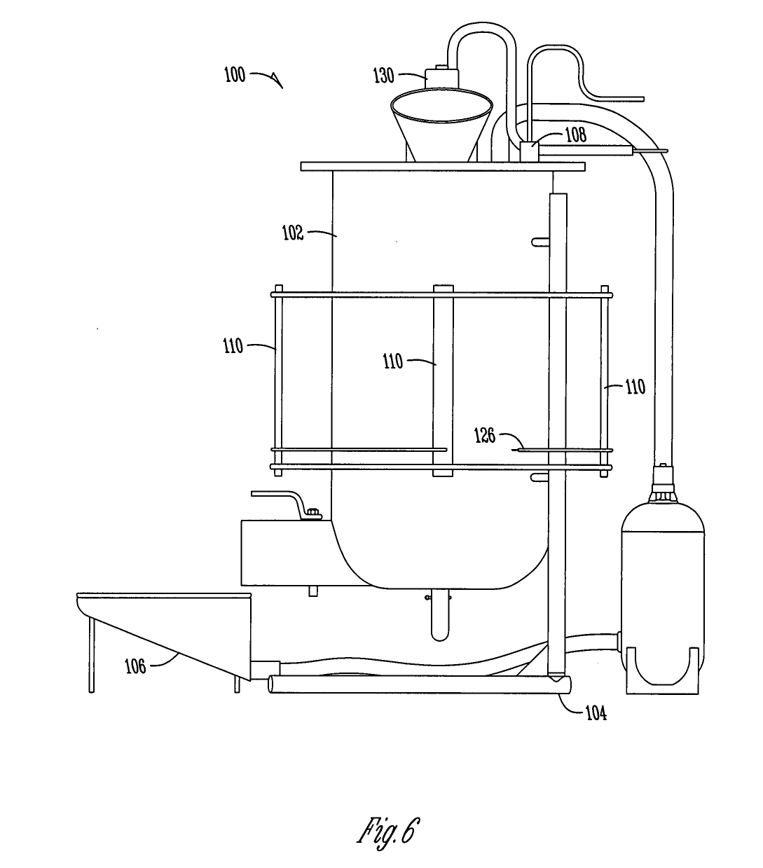

FIG. 6 is a view of one embodiment of a magnetic fermenter 100. The magnetic fermenter 100 is a modified New Brunswick Scientific (NBS) BioFlow 110 Fermenter. There is a fermentation tank or vessel 102 which is supported by a support 104. A sieve 106 is shown which may be used for straining. A gaussmeter 108 is shown which is used for measuring magnetic field associated with the fermentation process. Direct current (DC) controlled electromagnets 110 are positioned around the fermenter for applying the magnet field. A motor 130 is used to drive the stirring or agitation of the agitator within the fermentation tank or vessel 102.

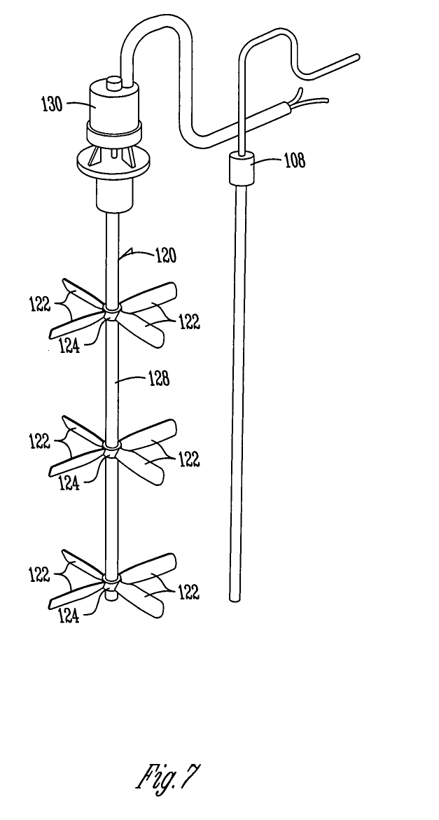

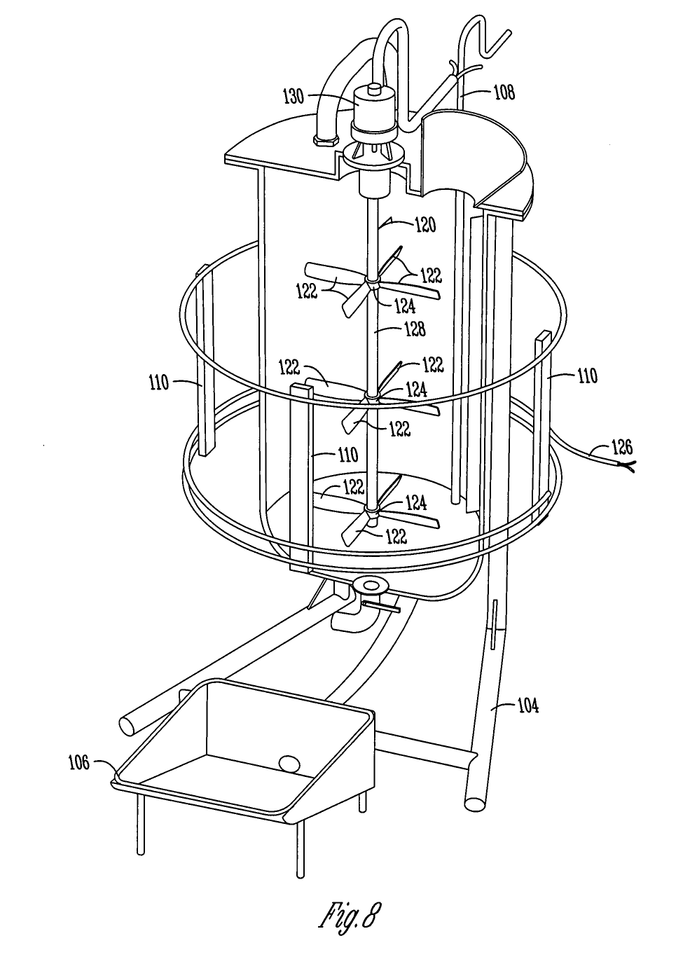

FIG. 7 illustrates the agitator or stirrer 120 which is placed within the fermentation tank or vessel 102. The agitator 120 includes a shaft 128 and impellers 121 with paddles or blades formed around annular member 124 which is positioned around the shaft 128. A submersible gaussmeter 108 is also shown. The submersible gaussmeter provides for measuring the magnetic field during the fermentation process. Although a gaussmeter or other magnetic field sensor is useful, particularly in experimental designs, to sense the magnetic field, such a sensor is not required once the effect of the magnetic field on a fermentation is established. A control system may still determine adjustments in the magnetic field to be made in various ways such as from production curves and then the magnetic field may be adjusted accordingly. FIG. 8 is a sectional view of the tank to show placement of the agitator 120 and the submersible gaussmeter 108 within the fermentation tank or vessel 102.

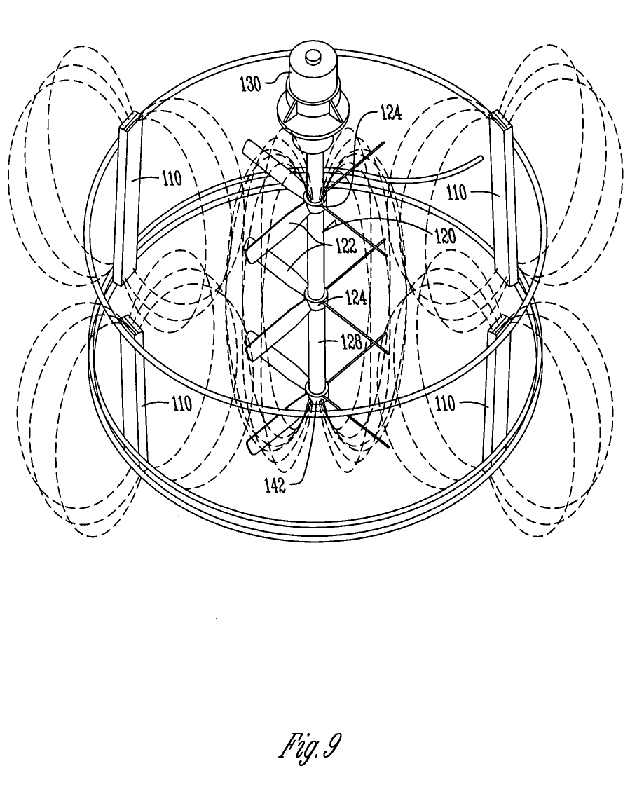

FIG. 9 is a representation of the magnetic fields applied. Note that a substantially monopole magnetic field can be applied to the contents of the vessel in this configuration. Depending upon the effect desired, the monopole magnetic field may be positive or negative. A variable DC power supply may be used to adjust the strength of the magnetic fields applied. Where permanent magnets or a DC power supplied electromagnet is used, the magnetic fields are static in nature. Static magnetic fields are generally magnetic fields that do not vary with time (frequency of 0 Hz). They are created by a permanent magnet or by the steady flow of electricity, for example using direct current (DC).

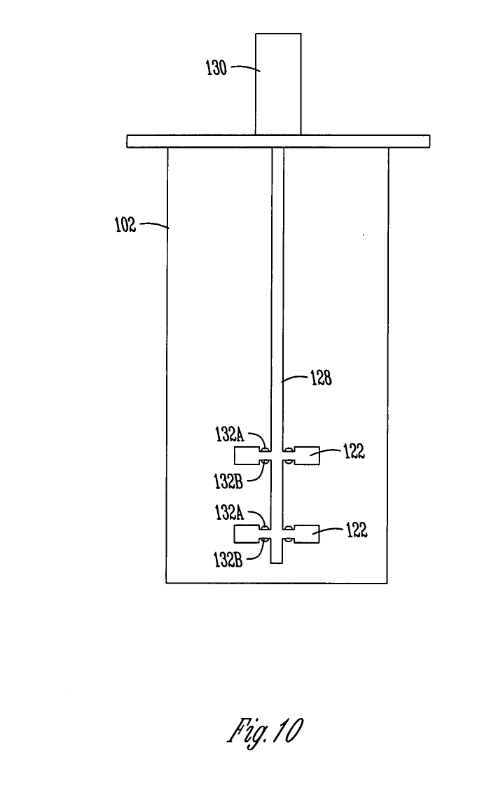

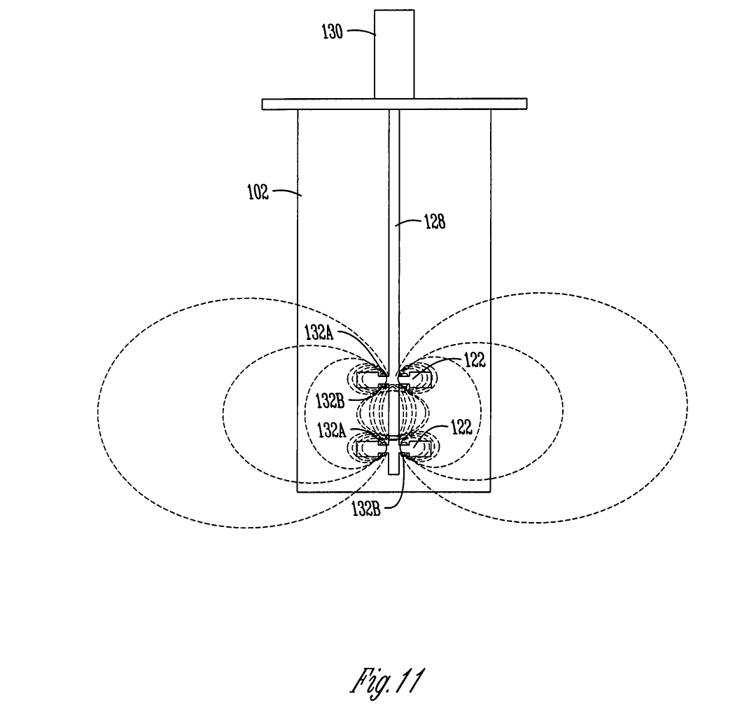

FIG. 10 illustrates another embodiment of the present invention. In the embodiment shown in FIG. 10, four magnetic rings were placed around the rod 128 of the agitator, with permanent magnets 132A, 132B sandwiching each impeller 121. Each of the permanent magnets 132A, 132B is formed of Neodymium-Iron-Boron grade-40. Modeling the motor, shaft, and paddles as 316 stainless steel in Finite Element Method Magnetics (FEMM) software resulted in the magnetic field flux line plots of FIG. 11 where there is a concentrated magnetic field moving “up” or from “south” to “north” near the shaft 128 while a larger and more diffuse field moves “down” or from “north” to “south.” Of course, by changing the orientation of the permanent magnets, a reversal of the flux directions may be achieved. Thus, in this embodiment, a magnetic field is applied using permanent magnets positioned within the fermentation vessel. It was also observed that for a similar setup, where the impeller 121 includes an annular member 124 of a soft metal to connect the blades or paddles, this soft metal may become magnetized during a fermentation process, and thereby provide similar results to the permanent magnets, even if the permanent magnets are not present in a subsequent trial.

Thus, as previously explained, the magnetic field may be applied to the contents of the fermentation tank from outside of the fermentation tank. Alternatively, the magnetic field may be applied to the contents of the fermentation tank by placement of the magnets within the fermentation tank. In addition, the magnetic fields may be generated in various ways including through the use of permanent magnets and/or electromagnets.

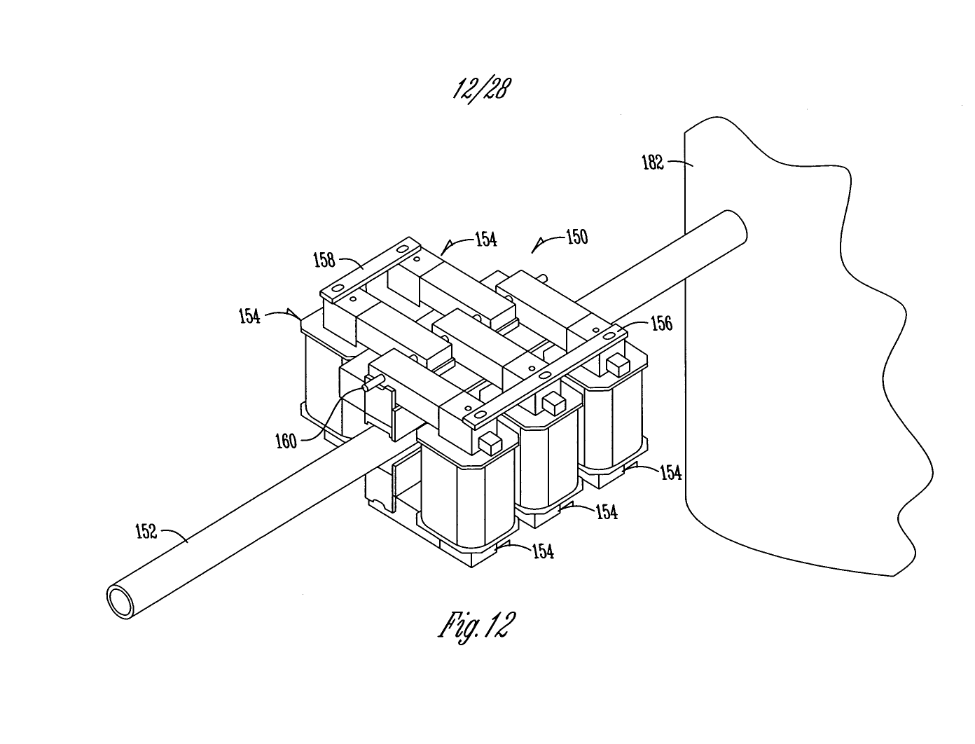

The present invention also contemplates that instead of creating the magnetic field within the tank, the magnetic field may be created to affect the moving fluid within piping or staging connected to the tank or otherwise associated with the fermentation process. By “fluid” it is intended to mean the fluid mixture, including the medium and other material present with the medium. For example, FIG. 12 illustrates a fermentation vessel or tank with a conduit or pipe 152. The pipe 152 may be a part of a recycle stage associated with the tank such as may be used in commercial production. The magnetic field may be applied to fluid within the pipe 152 through which the fluid flows back into the fermentation tank 102 during the recycle stage. Such a location for the magnets may be particularly advantageous for a number of reasons. One reason is that this configuration can be used to conveniently retrofit a commercial production process as it does not require modification of the tank itself. Another reason is that the cross-sectional area of the pipe 152 is significantly less than the fermentation tank 102, so the magnetic field created on an outer surface is more uniform and requires less energy to create.

The device 150 is modular in design, and includes one or more magnetic modules 154. Each magnetic module includes an electromagnet as will be explained in greater detail. A tie rod 160 extends through each of the magnetic modules with a hex nut 162 on each end of the tie rod 160. In addition, module retention straps 156, 158 are used to secure the magnetic modules to one another. Although a configuration of five magnetic modules 154 is shown in FIG. 12, any number of magnetic modules may be used in order to obtain a desired affect on the fermentation process. The number of magnetic modules may vary according to rate of fluid flow or other factors. FIG. 13 provides a top view of the device 150. FIG. 14 provides a front view of the device 150. There is a lifting eye 160 for each magnetic module.

The magnetic modules 154 are further illustrated in FIG. 15-FIG. 17. Each magnetic module includes an electromagnet coil assembly 170. The electromagnetic coil assembly 170 includes a core upon which conductive wire is wound into a number of turns. Electromagnetics are generally well known devices and the magnetic field created by an electromagnetic has known relationships which can be used in the design process so that appropriate number of coil turns, and length of coil can be used to create a desired magnetic field. Thus, for example, more current through the coil increases the magnetic field, more turns of wire increase the magnetic field, and the magnetic core material used affects the magnetic field. The core is generally u-shaped and formed of a paramagnetic or ferromagnetic material. The core concentrates the magnetic field to provide a stronger magnetic field.

There is a gang housing 184 adjacent or otherwise proximate the electromagnet coil assembly 170 which includes an electromagnetic coil 171. A magnetic nose cover 170 and a stop plate are also shown. Pole pieces 176 are both provided on the same side of the device 154. A magnetic retention cover 178 is placed over each polepiece 176 to maintain it in place. As best shown in FIG. 17, a magnet transfer box is also present. Each magnetic module 154 is thus generally U-shaped with a base 169 and legs 167 extending from the base 169 with an electromagnet at the base 169 and the pole pieces 176 near the legs 167. Returning to FIG. 12, note that the magnetic modules are 154 arranged in an alternating fashion such that the base of each magnetic module 154 is opposite the base of any immediately adjacent magnetic module in a zipper-like fashion.

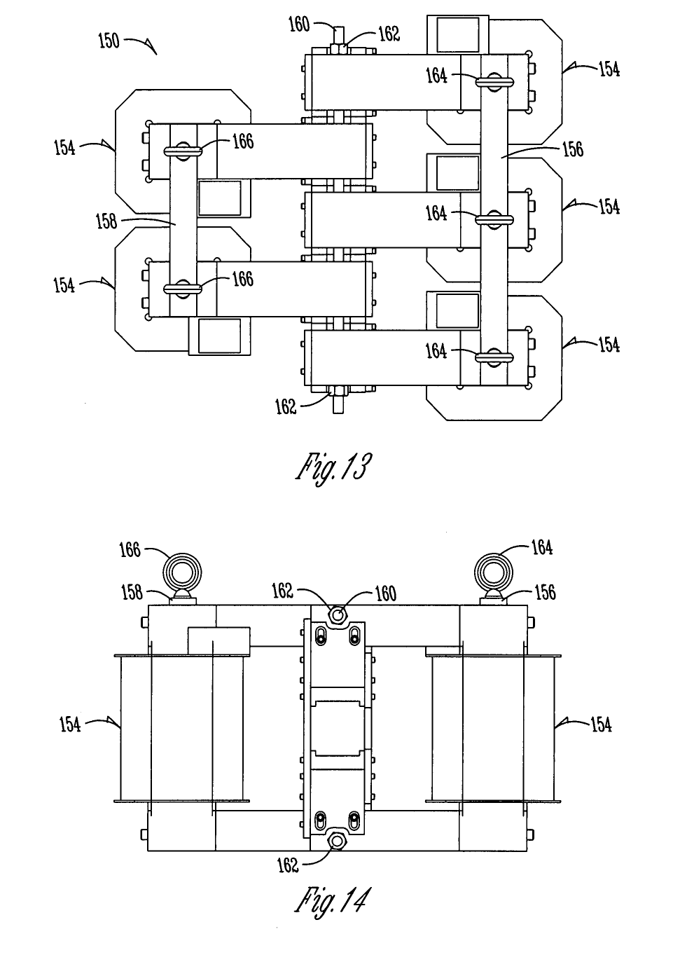

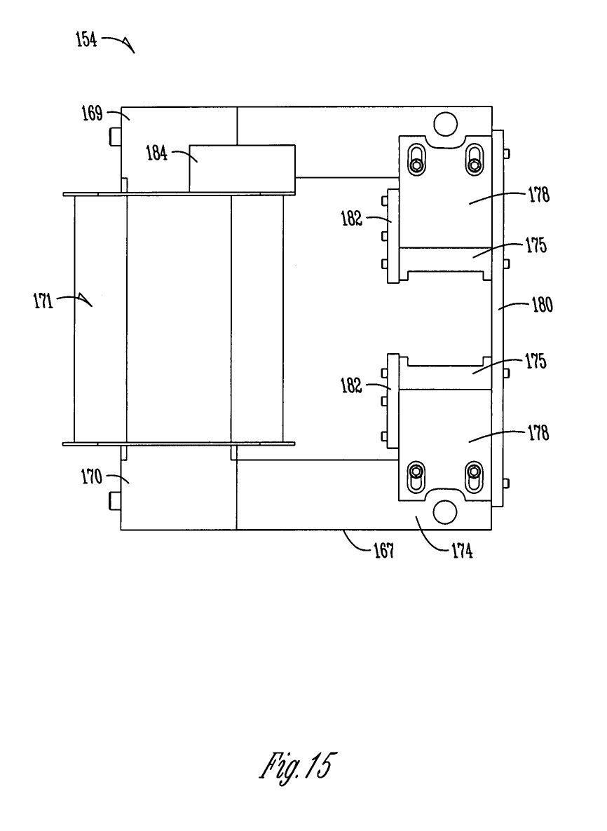

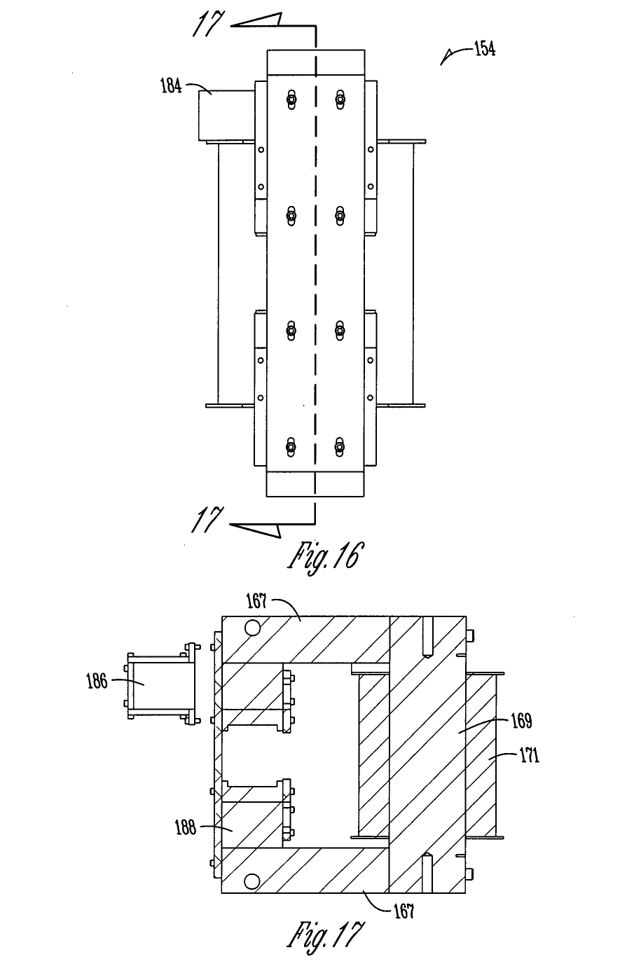

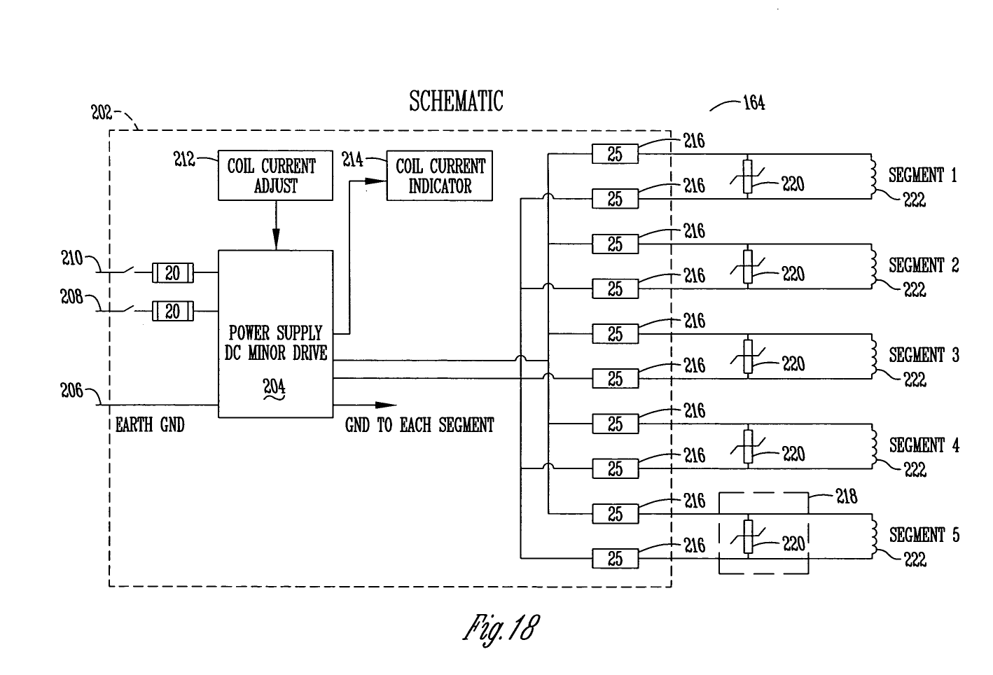

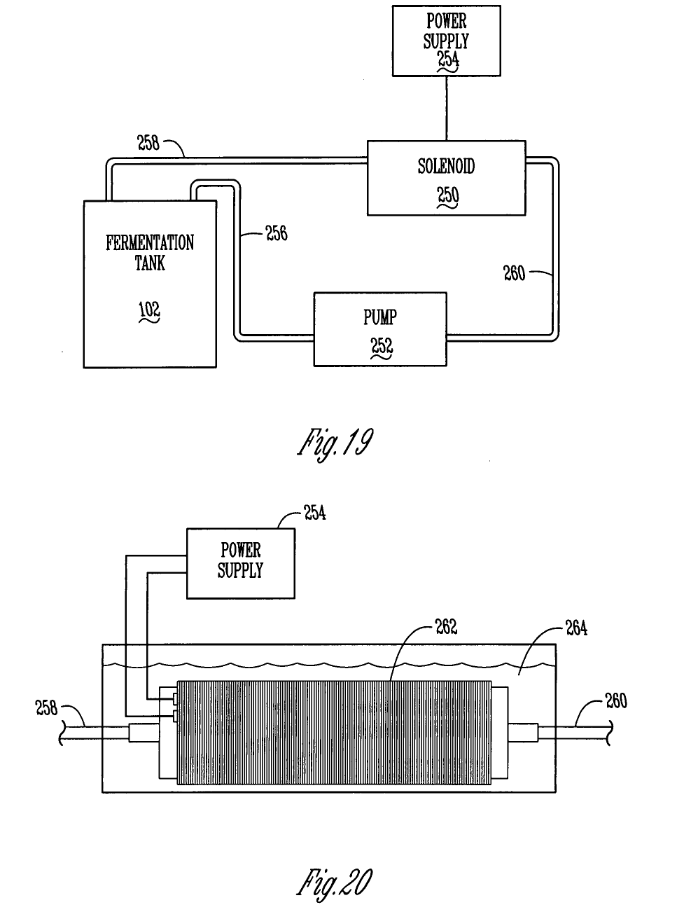

FIG. 18 illustrates an electrical schematic for the device 150. A portion 202 of the circuit 200 may be placed within a control box. The circuit includes a DC power supply 204. The DC power supply 204 is electrically connected to an earth ground 206. In addition, the DC power supply 204 is electrically connected to lines 208, 210. A coil current adjust control 212 is electrically connected to the power supply. The coil current adjust provides for adjusting coil current, such as through varying the setting of a potentiometer or otherwise. The coil current adjust control 214 may also be electrically connected to a coil current indicator 214. In operation, control of the coil current may be performed manually or by an intelligent control. The DC power supply 204 is electrically connected through fuses 216 before reaching each coil. A potentiometer 220 with an independently adjustable setpoint may be housed within a segment interconnect box 218 and used to adjust the setpoint. It is observed that the embodiment of the device of FIG. 12 through FIG. 18 has applications beyond the fermentation process and may be used in other applications where a magnetic field is desired, especially where the magnetic field is to be applied to a conduit or the contents thereof.

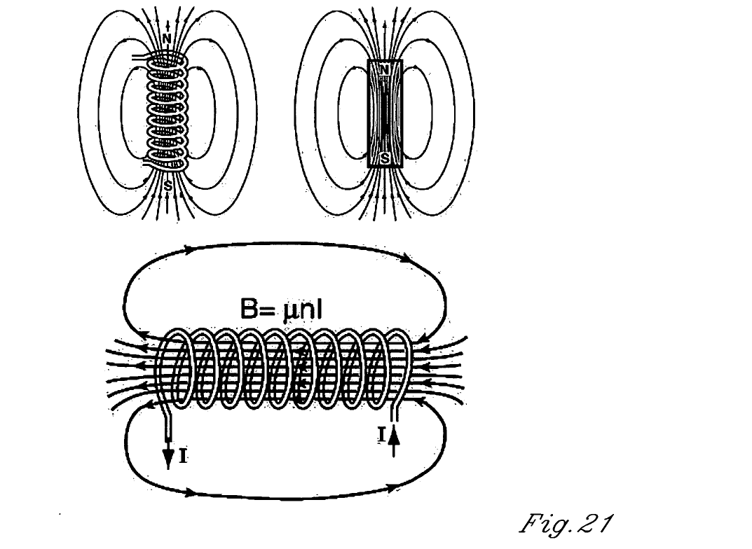

Another method for creating the magnetic field is to use a solenoid device 250 as shown in FIG. 19-21. In FIG. 19, a fermentation tank or vessel 102 is shown. A conduit 256 fluidly connects the fermentation tank 102 to a pump 252. The pump may be a Millipore pump (with no air intake). The pump 252 is fluidly connected to piping running through the solenoid 250. The solenoid device 250 is powered by a power supply 254. Conduit 256 fluidly connects from the output of the solenoid device 250 back into the fermentation tank 102.

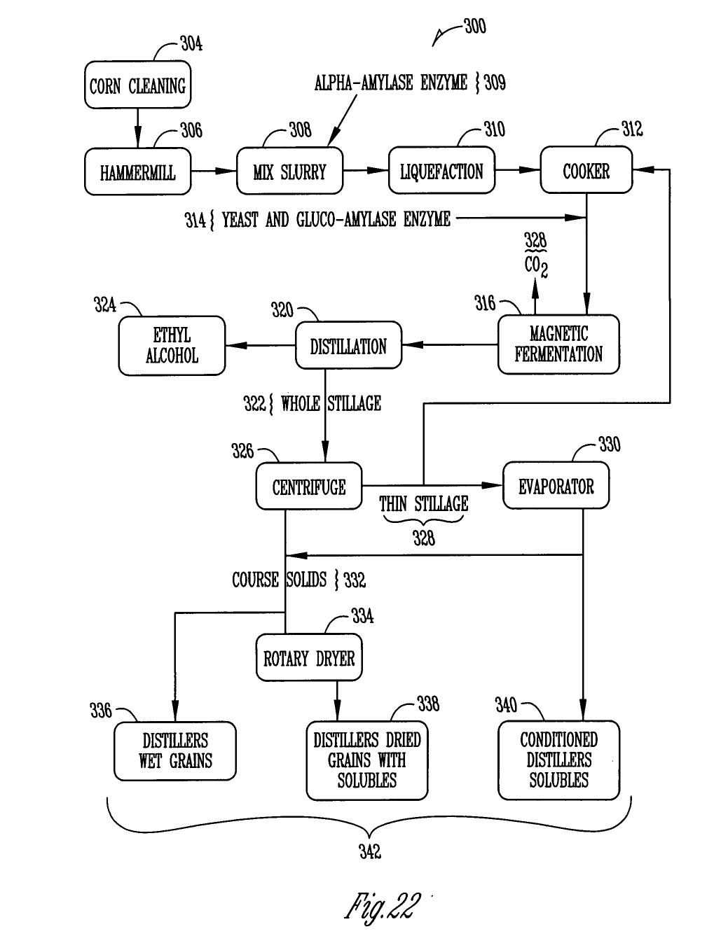

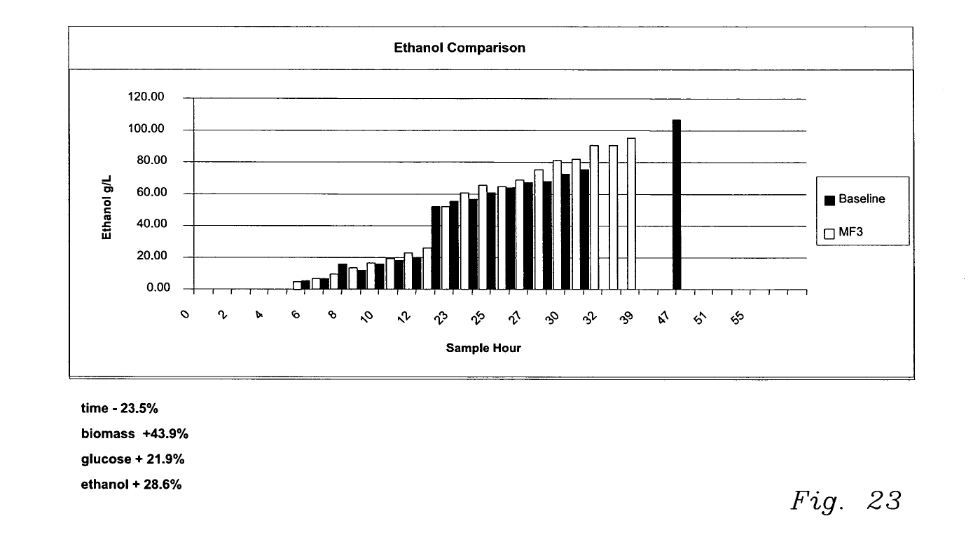

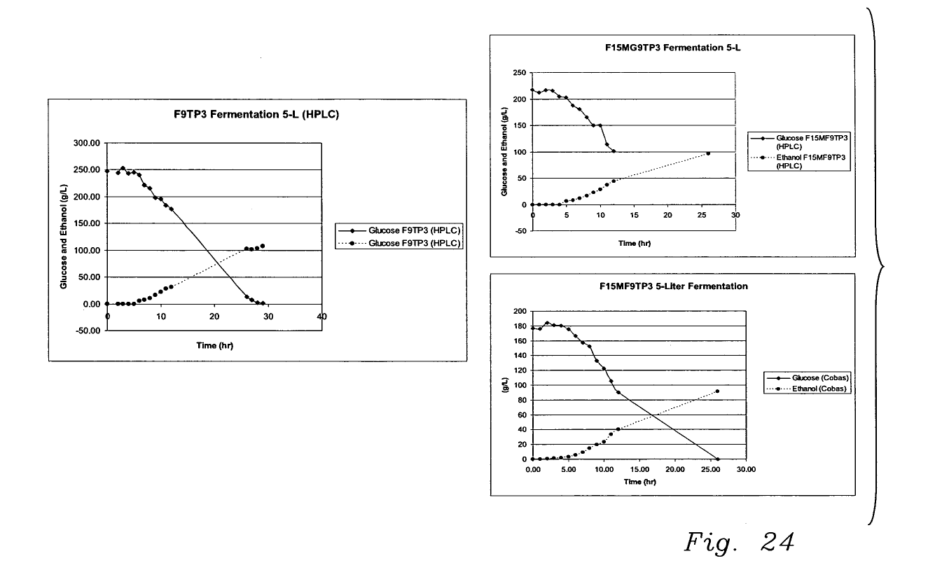

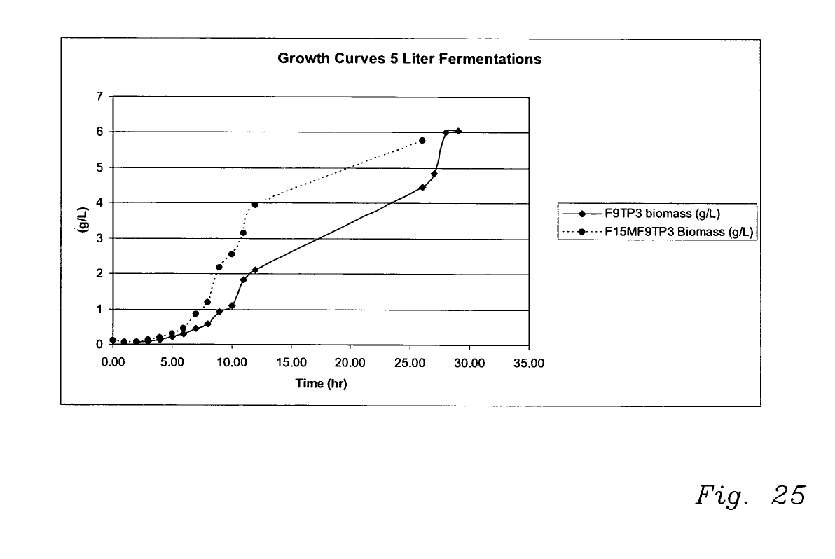

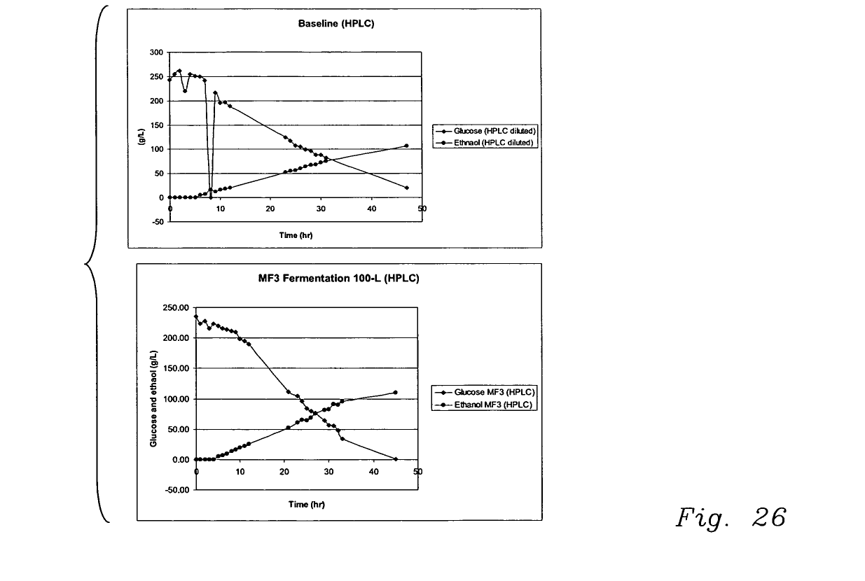

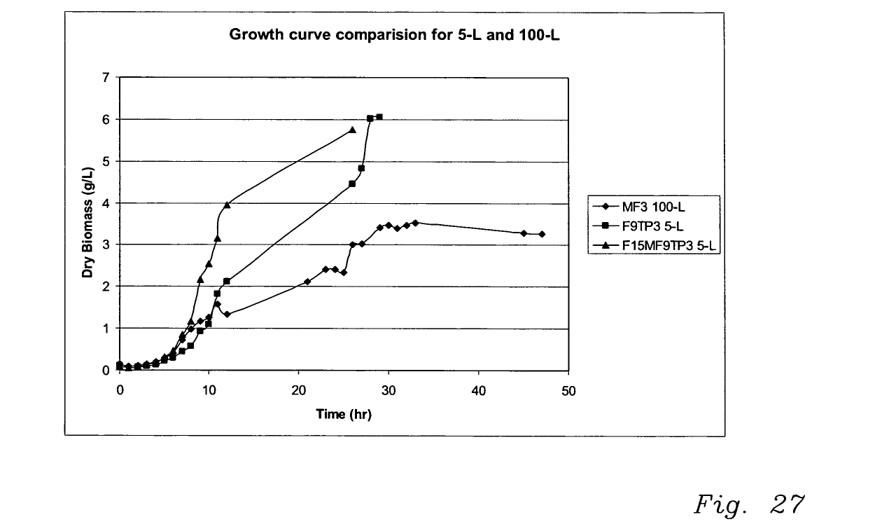

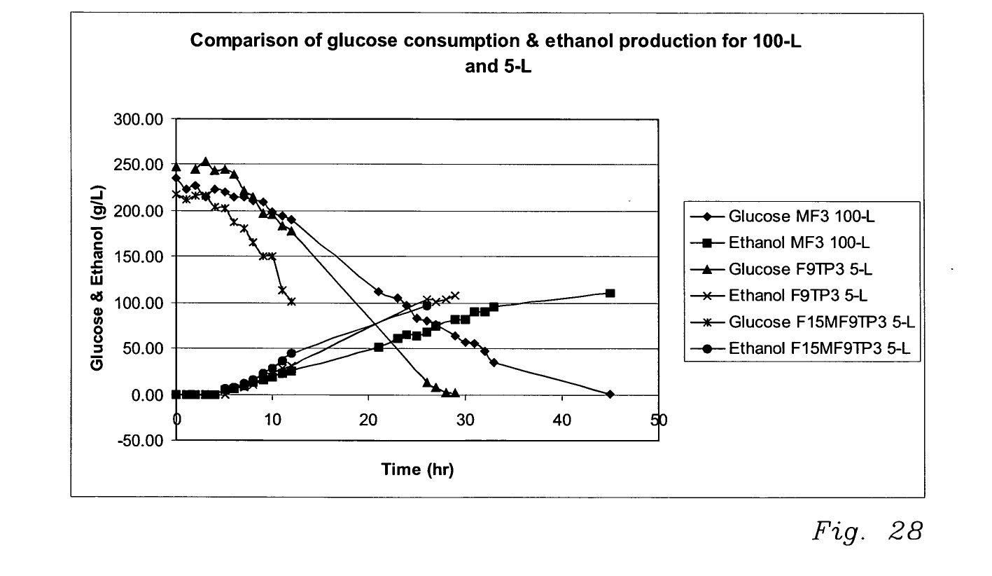

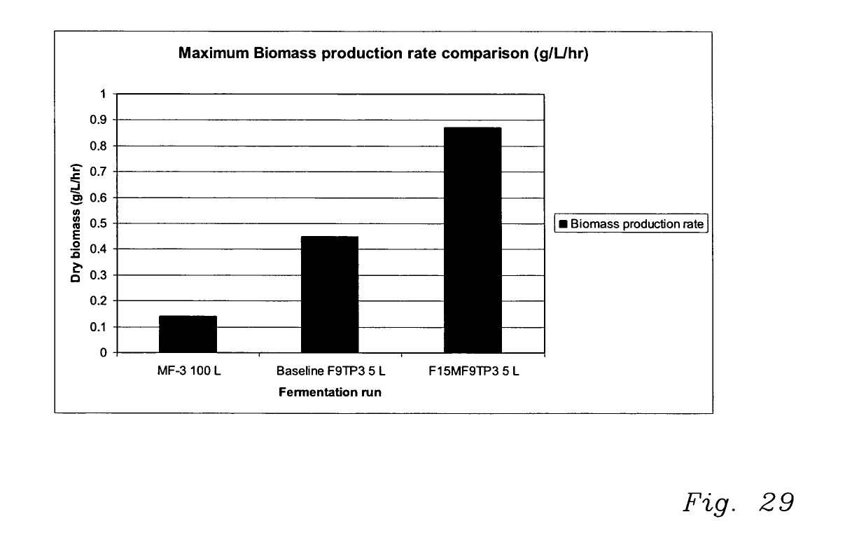

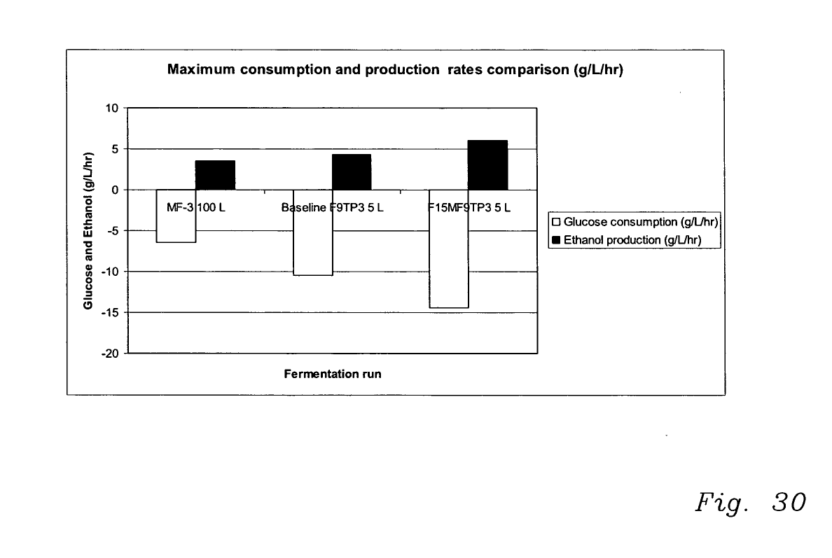

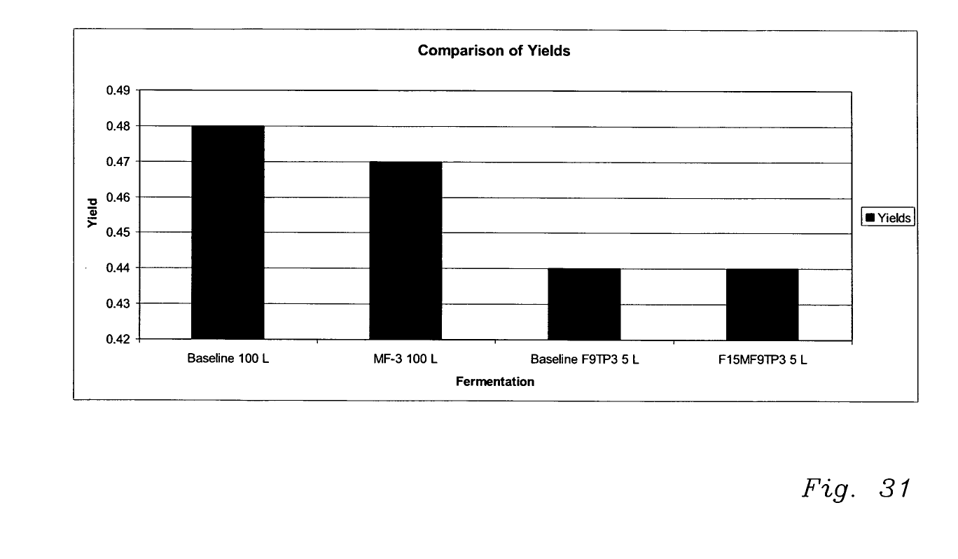

FIG. 20 illustrates the solenoid device 250 in more detail. As shown in FIG. 20, the solenoid device 250 includes a coil 262 which is placed within a mineral oil bath 264 for cooling. In operation, the fluid (including enzymes, bacteria, yeast, solids, medium, etc.) passes through the magnetic field generated by the coil 262. Without being bound to a theory of operation, it is believed that the electromagnetic field generated by the coil 262 when of the proper polarity simulates the tetrahedral water molecule structure to release oxygen and hydrogen and also to stimulate the metabolic activity of the microorganisms, increasing cell division.

FIG. 21 illustrates the magnetic fields generated from the coil 26. The magnetic field shown in concentrated into nearly uniform field in the center of the solenoid. The field outside is weak and divergent.

Although various embodiments have been provided showing how magnetic fields may be created, the present invention contemplates that any number of devices or methods may be used. One alternative design is to use an electromagnetic formed from a high energy coil. However, when a high energy coil is used, cooling of the coil may also need to be performed, such as through placement of the coil in mineral oil, periodically switching the electromagnet off or otherwise cooling the coil. Another alternative design is to place plastic casings containing permanent magnets and coil around the vessel. In such an embodiment, permanent magnets may be placed in a plastic casing. Subsequently, coils may be wrapped around the plastic casing and a second casing may be placed around the first casing and the coils, thus creating an electromagnetic device whose magnetic field affects the complete volume of the fermenter.

### III. Effects on Fermentation

In accordance with the present invention, methods and apparatus are described for the fermentation of a biological material, such as a fermentable carbohydrate, in a medium using a magnetic field to produce a fermented product, such as ethanol. Advantageously, use of the methods of the present invention increase the yield and/or production rate of a fermented product. Experiments subjecting Ethanol Red yeast and glucose to a positive magnetic field results in the yeast multiplying twice as fast as yeast not exposed to a magnetic field as well as producing ethanol 1.4 times faster than conventional fermentation methods. Use of fermentation methods of the present invention will impart significant savings to the industry. Without wishing to be bound by this theory, it is contemplated that the use of a magnetic field and the ability to modulate the magnetic field during fermentation confers several advantages. These include but are not limited to any of the following: speeding-up the dissolving of the oxygen in the medium/water, disrupting the water's hydrogen bonding to generate unstructured water with fewer hydrogen bonds to provide a more reactive environment, and increasing the rate of enzyme or bacteria's cell division. Importantly, cells convert a larger faction of a sugar substrate towards cell mass production as the amount of oxygen available to the cells speeds-up (increases). It has been observed that trace oxygen can serve as a nutrient during the anaerobic fermentation of sugars, allowing the fermentation rate to increase as more cells are produced.

In one aspect, the invention provides for a method of producing a fermented product that includes subjecting a biological material in a medium to a magnetic field to affect fermentation of the biological material.

### A. Biological Materials

In one aspect, the biological materials include but are not limited to sugars, plant extracts, inulin, biomass, biomass containing cellulose, hemicellulose, lignin, carbohydrates, starches, egg shell membranes, mash, pharmaceutics, nutrients, biominerals, fruit sugars, cane press juice (high test molasses), corn/grain based starches/sugars, cellulose hydrolysate, any source of glucose, sucrose, maltose, fructose, sucrose fractions, betaine fractions, xylose fractions, residual fractions, recycled fractions, betaine, xylose, galactose, rhamnose, mannose, xylonic acidbetaine, sugar alcohols, monosaccharides, hexoses, pentoses, xylose arabinoses, lignosulphonates, oligosaccharides, complex structural polymers containing cellulose, pectins, lignins, lignocelluloses, lignin-celluloses or cellulose-containing plants, the products of cellulose-containing plants, stems, hulls, husks, cobs of plants, unprocessed plant materials, lawn clippings, leaves, fibers, pulps, hemps, sawdusts, newspapers, agricultural crops, grasses, cotton, cotton stalks, corn stalks, corn cobs, wheat straws, oat straws, rice straws, cane sugars (bagasses), soybean stalks, peanut plants, pea vines, sugar beet wastes, sorghum stalks, tobacco stalks, maize stalks, barley straws buckwheat straws, quinoa stalks, cassayas, potato plants, legume vines stalks, vegetable inedible portions, weeds, vines, kelps, flowers, algaes, bioenergy crops, trees, agricultural residues, wood residues, cellulosic fiber fines, waste papers, commercial waste products containing cellulose, paper, cotton clothes, bagasse wallboards, wood products, trees, shrubs, corn, husks, municipal solid wastes, waste papers, yard wastes, biomass high in starch, grains, fruits, vegetables, branches, bushes, canes, forests, herbaceous crops, barks, needles, logs, roots, saplings, short rotation woody crops, switch grasses, vines, hard soft woods, or organic waste materials generated from agricultural processes including farming and forestry activities, specifically including forestry wood waste and any biological material suitable as a substrate for fermentation.

One skilled in the art would be knowledgeable in the selection of the biological material for use in producing a particular fermented product. For example, complex saccharide substrates may be used as a starting source for depolymerization and subsequent fermentation for use in simultaneous saccharification and fermentation process.

### B. Medium

In one aspect, the medium may include any medium such as water, a broth, a nutrient solution or any other medium that comes in contact with the biological material and is capable of facilitating the fermentation process. The term “broth” includes a solution as well as a suspension. The broth may be largely inorganic and include any number of minerals in solution to provide the major nutrient ions such as sodium, potassium, phosphate, sulfate, magnesium and iron and optionally an organic chelating agent to keep iron from precipitating. The absolute concentrations of the nutrients in the broth are not critical as long as they are present in adequate amounts for the microorganisms to grow but not so high as to inhibit growth. Broths include standard bacteriological growing media which can be modified in any standard manner. Typical nutrient solutions, which are well known to skilled artisans, may include minimum nutrient broth, yeast extract, corn steep liquors and the like. The appropriate medium to use in conjunction with a particular fermentation process may be determined by one skilled in the art and the medium prepared from any number of routine protocols, such as the one described in Example 1.

### C. Microorganism

In one aspect, the method includes the use of a microorganism, such as yeast, bacteria, or fungus or combinations thereof to facilitate the conversion of the biological material to a fermented product. More than one biological material or microorganism may be placed within the apparatus depending on the type of fermentation process to be carried out. Any suitable microorganism or combination of microorganisms which ferments a biological material to produce a fermented product such as ethanol may be used with the methods of the present invention. “Conversion” includes any biological, chemical and/or bio-chemical activity which produces a fermented product, such as ethanol or a byproduct from the biological material, such as biomass. Suitable microorganisms for use with the methods of the present invention include yeast or bacteria that consume sugars derived from the hydrolysis of biomass. See U.S. Pat. No. 6,927,048, herein incorporated by reference in its entirety.

Genetically engineered or natural strains of microorganisms maybe used in the conversion of the biological material to a fermented product, for example, bacteria, such as Zymomonas mobilis and Escherichia coli; yeasts such as Saccharomyces cerevisiae or Pichia stipitis; and fungi that are natural producers of the fermented product, such as ethanol. The term microorganism further encompasses mutants and derivatives, such as those produced by known genetic and/or recombinant techniques, for example, those that contain pyruvate decarboxylase and/or alcohol dehydrogenase genes, those that have been produced and/or selected on the basis of enhanced and/or altered the production of the desired fermented product, e.g. ethanol.

In one aspect of the invention, an enzyme preparation may be used with the methods described herein. The enzyme preparation may include any enzyme that ferments a biological material into a fermented product. The preparation may be contain isolated or recombinant enzymes. See for example, U.S. Pat. No. 7,226,776, herein incorporated by reference in its entirety. In one aspect, a microorganism and an enzyme preparation may be used in combination in accordance with the methods of the present invention.

In one aspect, microorganisms and/or enzyme preparations, concentration of the microorganisms and/or enzyme preparations, and conditions (e.g. pH, fermentation medium, levels of nutrients, and temperature) are selected in accordance with standard techniques and may be optimized for both yield and efficiency (for example, ethanol production rate). Suitable microorganisms are well-known and commercially available.

In one aspect of the invention, the temperature of the medium comprising the biological material may be monitored using a temperature sensor and modulated in accordance with the desired operating conditions. Typically, fermentation is carried out at a temperature within the range of from about 25° C. to about 40° C., preferably within the range of from about 30° C. to about 35° C. In one embodiment, the medium having the microorganism and biological material may be heated to optimize production of the fermentation product or alternately cooled to prevent the temperature from rising to a temperature that would be stressful to the microorganisms, for example, kill them.

As described herein, the optimum pH may achieved through monitoring the pH of the medium using a pH sensor and modulating the pH by modulating the magnetic field. The optimum pH can vary from about 4.5 or lower for some microorganisms such as some yeasts, up to about 6.0 to about 6.7 or higher for other microorganisms, such as recombinant organisms. Determining the optimum pH for any given microorganism is well within the routine skill of the skilled artisan.

### D. Products

Using the methods and apparatus of the present invention, the biological material may be fermented to produce any number of products. In one aspect, the fermented product may include any fermented product or by-product such as ethanol, citric acid, butanol, isopropanol, lactic acid, collagen, pharmaceutics, such as antibiotics, for example, penicillin G, penicillin V and Cephalosporin C. (See K. Matsumoto, Bioprocess. Techn., 16, (1993), 67-88, J. G. Shewale & H. Sivaraman, Process Biochemistry, August 1989, 146-154, T. A. Savidge, Biotechnology of Industrial Antibiotics (Ed. E. J. Vandamme) Marcel Dekker, New York, 1984, or J. G. Shewale et al., Process Biochemistry International, June 1990, 97-103), and the like.

### E. Magnetic Field

A determination is made with respect to whether a positive or negative magnetic field should be applied to the biological material. A positive magnetic field and a negative magnetic field will generally have the opposite effects on the fermentation process. Consideration should be given to whether the fermentation reaction occurs in an acidic or alkaline medium. For example, for reactions that take place in an alkaline (basic) medium such as oxidoreductase catalysis, a negative static magnetic field should be applied. Conversely, for reactions that take place in an acidic medium, such as fermentation, a positive static magnetic field should be applied. Without wishing to be bound by this theory, it is believed that a positive static magnetic field increases the activity of transferases, hydrolases or both. Both oxidation phosphorylation and fermentation catalysis are energized by static magnetic fields; however, the reactions are energized by opposite magnetic poles. It is believed that oxidation phosphorylation is energized by a negative static magnetic field in an alkaline-hyperoxic medium, whereas fermentation is energized by a positive static magnetic field in an acid-hypoxic medium.

Any suitable apparatus may be used in conjunction with the methods of the present invention so long as the apparatus can be used to ferment the biological material and a source of a magnetic field can be applied to the biological material. Any suitable apparatus can be used, for example, batch, fed-batch, cell recycle, continuous process or multi-step bioreactors. Accordingly, any suitable technique may be used to expose the biological material to the magnetic field, for example, a magnetic field may be applied internally or externally with respect to the apparatus, passing through the apparatus if needed to reach the biological material placed within the apparatus. In one aspect, the magnetic field may be placed to take advantage of the circulating biological material as it is believed that aerating or suspending the microorganisms may improve the magnetic effect.

The magnetic field may be applied using any source of such radiation, for example, permanent magnets or electromagnets. In general, the magnetic source is applied to the biological material in the apparatus for an appropriate time and at an appropriate level to affect fermentation. Depending on the effect desired, a negative field may also be applied. In one aspect, the range of the applied magnetic field is from about 2000 to about 3000 Gauss and it is preferred that the field is generally uniform, although it is recognized that some variation may be present. In one aspect, the magnetic field is from about 2200 to about 2400 Gauss. In one aspect, a DC electromagnetic “monopole positive” flux density of about 2200 to about 2400 Gauss is applied on the medium or fermenter's fluid/solids. In one aspect, the medium with the biological material/fluid is passing through a pipe at no more than 0.1 to 5 seconds at any given time. In another aspect, the medium with the biological material/fluid is passing through a pipe at less than 20 minutes of the fluid cycle. Any level of magnetic field, length of exposure to the magnetic field or flow rate of the medium/fluids may be used so long as the fermentation reaction is able to take place. According to the present invention, a method of fermenting a biological material includes contacting a medium with a biological material and a source of a magnetic field for a time sufficient for the fermentation to occur. The present invention contemplates providing the magnetic field for a first time period, and then turning the magnetic field off for a second time period. For example, the magnetic field may be turned on and off every 30 seconds. Of course, other time periods may be used, and the first time period during which the magnetic field is applied need not be the same duration as the second time period where the magnetic field is turned off. It is preferred that any amount of medium/water/solids or fluid is not exposed continuously to a magnetic field for more than 30 seconds.

Optimal levels of magnetic fields may be determined. It has been observed that in some instances, applying magnetic fields greater than 3000 Gauss decreases ethanol production from yeast. As previously explained, a magnetic field sensor may be coupled to a feedback arrangement and a controller for modulating the magnetic field until the magnetic field is at or above a predetermined magnetic field value. The magnetic field may be monitored and compared to a previous level of the magnetic field, for example, using a submersible Gauss meter and feedback loop. In one aspect, one or more detectors are used to measure the intensity of the magnetic field. In one aspect, the detector is placed within the apparatus. Alternately, the detector may be placed external to the apparatus opposite the source of the magnetic field depending on the strength of the field. The intensity of the field may be modulated to achieve a given parameter. The adjustment may be automated or manual. The magnetic field may be monitored and compared to a previous level of the magnetic field, for example, using a Gauss meter and feedback loop.

According to one aspect of the invention, the methods include modulating the level or intensity of the magnetic field during the fermentation process. As used herein, the term “modulate”, “modulates” or “modulating” refers to a change, i.e. an increase or decrease in the magnetic field.

The biological material is exposed to medium comprising one or more sources of the appropriate magnetic field and optionally a pH sensor. The medium is exposed to the source of magnetism for a time sufficient for the reaction to take place. As the reaction occurs, the pH of the medium will change, Accordingly, in one aspect, the methods include using one or more pH sensors to detect a change in the pH of the medium as the fermented product is produced. For example, as an acidic product is produced, the medium becomes more acidic and the pH decreases. Conversely, when an alkalinic product is produced, the medium becomes more basic and the pH increases. The change in the pH of the medium may be detected using any number of methods. The differences in change of pH may be compared to a control that is not subjected to a magnetic field.

### F. Monitoring pH

As discussed previously, the pH of the medium may be assayed to determine whether a positive or negative magnetic field should be applied to the biological material. The biological material is exposed to medium comprising one or more sources of a magnetic field and optionally a pH sensor.

Accordingly, in one aspect, the method includes determining the pH of the medium comprising a biological material so that the pH can be modulated using a magnetic field. If the fermentation takes place in an acidic medium, then the pH can be monitored and, if desired, regulated to maintain an acidic pH, for example, within a desired pH range. For example the pH of the medium containing the biological material may be adjusted, for example, to a pH of about 3 to about 4.5 when fermenting glucose to produce ethanol. As a fermented product such as ethanol is produced, the medium becomes more basic. If the fermentation takes place in a basic medium, then the pH can be monitored and, if desired, regulated to maintain a basic pH.

The medium may be evaluated for a change in pH, for example, using a pH sensor. The sensor may operate continuously or at frequent time intervals to monitor the pH. Prior to any detection, an initial pH level of the medium may be determined. In one aspect, the change in the pH may be monitored at various time points for example, at an initial starting point and then at various time points thereafter and compared to the previous pH reading. Time points may vary from hours to days depending on the criteria of the experimental design and the type of fermentation product being produced. Such criteria include but are not limited to the amount of biological material in the medium, the amount of medium, the temperature and the type of microorganism. If a reading from the pH sensor detects an unacceptable pH level, the intensity of the magnetic filed can be modulated. Note that there is no need to add an acid or base to alter pH levels of the medium. These affects can be achieved in a chemical free manner.

In one aspect, a pH sensor is coupled to a feedback arrangement and a controller for modulating the magnetic field until the medium is at or above a predetermined pH. The change in the pH of the medium may vary depending on the medium and biological material present in the medium, the microorganism and the amount of fermented product produced. A variety of detectors such as a magnetic field sensor, pH sensor, temperature sensor, oxidation reduction potential sensor, ethanol or glucose sensor can be selected to provide a number of measurements for use in the methods and/or apparatus of the present invention, which measurements will depend on the type of fermentation reaction and the parameters being controlled.

The amount of biological material or fermented product produced by the methods of the present invention may be determined. The differences in the amount of biological material or fermented product may be compared to a control that is not subjected to a magnetic field to determine yield or efficiency for the fermentation methods. The yield or rate of fermented product produced using fermentation methods of the present invention may be determined and compared to the yield or rate relative to another fermentation method that does not use magnetic fermentation using, for example, qualitative, quantitative, or statistical evaluation.

As used herein, “yield” may include reference to the amount of fermented product produced, for example, the amount of fermented product produced (gr/l), such as ethanol or lactic acid, divided by the amount of biological material consumed (gr/l), such as glucose. One skilled in the art will be able to determine yield for a particular fermented product. For example, the medium may be removed from the apparatus to facilitate determination of the level of biological material or fermented product in the medium. Levels may be determined using for example High Performance Liquid Chromatography (HPLC), a Biochemistry Analyzer such as YSI 2700 (YSI Inc, Yellow Springs, Ohio) or Cobas Mira Biochemistry Analyzer (F. Hoffmann-La Roche, Ltd, Nutley, N.J.), or mass spectrometry. A “control” may comprise, for example: (a) medium that contains the same starting biological material but which has not been subjected to a magnetic field (b) a medium that does not contain a starting biological material. Thus, a “control” may be used to provide a reference point for measuring changes in pH, yield, or production rate, or concentration of biological material or fermented product when using the fermentation methods of the present invention as compared to more conventional fermentation methods.

The fermentation methods described herein can include any number of steps, for example, a feeding step where the biological material is broken down or consumed, a recycling or circulation phase, and a product recovery phase where the fermented product is recovered. Other products of cellulose-containing plants may be recovered using the methods of the present invention such as waxes, gums, oils, sugars, wood alcohol, agar, rosin, turpentine, resins, rubber latex, dyes, glycerol, etc.

Advantageously, at least some of the steps during the fermentation process can occur sequentially, continuously, or simultaneously. In one aspect, the method includes membrane filtration, for example, for use in a saccharification stage, byproduct recovery stage or fermentation stage to retain enzymes, carbohydrates, salts, or microorganism to enhance the rate of fermentation. In another aspect, membrane filtration may be used to recover byproducts produced in some fermentation processes such as glycerol, lactic acid and others and or to reduce the amount of solids going to an evaporator.

The fermentor used with the methods of the present invention is typically an anaerobic fermentor which may be continuous, batch fed, or simple batch. Carbon dioxide, which is byproduct of fermentation, can be removed continuously from the fermentor. If a continuous or batch fed fermentor is used, then optionally, on a continual basis, fluid having ethanol may be drawn off from the fermentor and treated to recover ethanol, for example, by evaporation and/or distillation. The ethanol concentration above which the fermenting organisms will decrease or cease production will depend upon the particular microorganism used. Accordingly, the fermented product produced by the methods and/or apparatus of the present invention may be recovered using any suitable method, for example, ethanol may be removed from the medium by evaporation or by membrane filtration technology.

### G. Monitoring an Oxidation Reduction Potential (ORP)

In one aspect, the fermentation method of the present invention includes monitoring the ORP of the medium using an ORP sensor. In one aspect, the ORP may be adjusted to optimize the ORP for the specific fermentation reaction. See U.S. Pat. No. 7,078,201 to Burmaster describing oxidant addition (such as air or oxygen sparging, peroxide etc), reductant substitution (such as ammonia with caustic), or reductant elimination (such as oxidation of sulfite) to adjust the ORP. Optimization of ORP is well within the skill of one skilled in the art.

### H. Effects

Without wishing to be bound by this theory, it is contemplated that the use of a magnetic field and the ability to modulate the magnetic field during fermentation confers several advantages. These include but are not limited to any of the following: speeding up the dissolving of oxygen in the medium/water, increasing the rate of enzyme or bacteria's cell division, and disrupting the water's hydrogen bonding to generate unstructured water with fewer hydrogen bonds to provide a more reactive environment. Importantly, cells convert a larger faction of the sugar substrate towards cell mass production as the amount of oxygen available to the cells speeds up which allows the fermentation rate to increase.

In one aspect, the methods of the present invention include increasing the yield of a fermented product. In another aspect, the methods of the present invention include increasing the production rate of the fermented product. In another aspect, the methods of the present invention include shortening the length of time to produce the fermented product. In another aspect, the methods of the present invention include increasing the growth rate of the microorganism.

As described previously, methods of the present invention may be used produce fermented products that have application in various industries, such as but not limited to food containing lactic acid such as yogurt, and alcohol in distilled beverages, such as potable beers, wines, and grain alcohols, as well as industrial and fuel alcohol such as ethanol, pharmaceutics, textile industries, and biodegradable plastics (Brown, S. F., 2003, Fortune, 148:92 94; Datta, R., et al., 1995, FEMS Microbiol. Rev. 16:221 231).

The methods of the present invention may be used in producing any number of products whose production employs fermentation. Examples include without limitation: cellulosic ethanol that uses hydrolysis of cellulose followed by fermentation of the generated free sugars; ethanol produced by methods such as the simultaneous saccharification and fermentation of a biological material such as glucose or inulin (Ohta et al. Production of high concentrations of ethanol from inulin by simultaneous saccharification and fermentation using Aspergillus niger and Saccharomyces cerevisiae. Appl Environ Microbiol. 1993 March; 59(3):729-33); polymeric hexose and pentose sugars in cellulose and hemicellulose; glucose, lactic acid produced by the fermentation of sugars and the like.

Accordingly, methods of the present may be used in the fermentation of a biological material to ethanol, the simultaneous saccharification and fermentation of a biological material to ethanol, and fermentation of a biological material to lactic acid.

### III. Fermentation of Sugar to Produce Ethanol:

In one embodiment, the methods and apparatus of the present invention may be used to ferment sugars or starches to ethanol using a positive magnetic field. In one aspect, a microorganism such as yeast is used to carry out the enzymatic conversion. In one aspect, microorganisms, concentration of the microorganisms, selection of biological materials and conditions (e.g. pH, fermentation medium, levels of nutrients, and temperature) are selected in accordance with standard techniques and may be optimized for both yield and efficiency (ethanol production rate). See U.S. Pat. No. 4,349,628 to English et al; see also U.S. Pat. No. 5,932,456 to Van Draanen et al., U.S. Pat. No. 4,400,470 to Zeikus et al; U.S. Pat. No. 5,000,000 to Ingram et al; U.S. Pat. No. 5,028,539 to Ingram et al; and U.S. Pat. No. 5,162,516 to Ingram et al, disclosing the conversion to ethanol of polymeric hexose and pentose sugars in cellulose and hemicellulose, all of which are incorporated herein by reference.

In one embodiment, the fermentation of ethanol from a biological material using a magnetic field may be part of a dry grind process, modified dry grind process or wet mill process. In one embodiment, the ethanol production facility utilizes grain as a starting biological material. In one embodiment, the grain is selected from the group consisting of sorghum, wheat, barley, oats and rice. The liquid medium processing stream can include heavy steep water, an uncooked slurry, a cooked mash, a liquefied mash, and (for a dry grind process) whole stillage, thin stillage and wet cake.

Those skilled in the art will appreciate, and readily accommodate, without undue experimentation, adjusting the magnetic field, concentration of the microorganisms, and conditions (e.g. pH, fermentation media, levels of nutrients, and temperature) for yield and efficiency all in accordance with the teachings disclosed herein.

It is understood that both the substrate and product of the ethanolic fermentation may inhibit the fermentation process or effect the fermentation rates. Accordingly, in one aspect, the method includes fermenting the sugar to ethanol and removing the resulting ethanol. The fermented product such as ethanol can be recovered using any suitable means, for example, by a ferment stripper, distillation or membrane technology. (See for example, U.S. Pat. Nos. 4,665,027 and 5,141,861, herein incorporated in their entirety) with gas stripping of ethanol from the broth, the vacu-ferm fermentation suggested by Ramalingam and Finn (1977), the coupled fermentation/distillation Biostil process developed by Alpha Laval and then acquired by Chematur Engineering (1994), etc.). Accordingly, ethanol may be separated using for example to remove ethanol as it is produced. The amount of ethanol produced may be analyzed, for example, by High Performance Liquid Chromatography (HPLC), a YSI 2700 Biochemistry Analyzer (YSI Inc, Yellow Springs, Ohio), Cobas Mira Biochemistry Analyzer (F. Hoffmann-La Roche, Ltd, Nutley, N.J.), or mass spectrometry.

In one aspect, methods of the present include a method for the Simultaneous Saccharification and Fermentation (SSF). In SSF, product inhibition of the cellulases can be avoided by conversion of the glucose into ethanol or other desired fermentation product. The SSF philosophy has been used for decades by the ethanol industry with starch enzymes. Research also shows that this concept works for the hemicellulase and cellulase enzyme systems. The Gulf Oil Company developed a method for the production of ethanol from cellulose using a yeast-based process termed simultaneous saccharification and fermentation (SSF) (Gauss et al. (1976) U.S. Pat. No. 3,990,944, herein incorporated in its entirety). Fungal cellulase preparations and yeasts may be used to produce ethanol from a slurry of the cellulosic biological material. Ethanol may be produced concurrently during cellulose hydrolysis.

### IV. Fermentation of Sugar to Produce Lactic Acid:

The invention also provides methods for the production of lactic acid by subjecting a biological material to fermentation using a magnetic field. In one aspect, the biological material is cellulose and hemicellulose. In another aspect, the method includes culturing a microorganism capable of fermenting a biological material under conditions suitable for the production of lactic acid. The method may further comprise the optional step of recovering include L(+)-lactic acid, 1,3-propanediol, 1,2-propanediol, succinic acid, ethanol and D(−)-lactic acid. See U.S. Pat. No. 7,098,009 to Shanmugam et al.

The configuration and components employed in any apparatus using fermentation methods may be coordinated with the application requirements, for example, the scale of the operation and amount of product desired. Various embodiments of the invention, including different configurations and utilizing diverse components for the generation of a magnetic field are possible.

This invention can be better understood by reference to the following non-limiting examples. It will be appreciated by those skilled in the art that other embodiments of the invention may be practiced without departing from the spirit and the scope of the invention as herein disclosed and claimed.

### EXAMPLES

The present invention is further defined in the following Examples, in which parts and percentages are by weight and degrees are Celsius, unless otherwise stated. The disclosure of each reference set forth herein is incorporated herein by reference in its entirety.

### Example 1

One fermentation protocol, referred to as MF3, includes carrying out fermentation of 5 L or 100 L in a bioreactor containing culture medium with 250 g/L dextrose, 20 g/L peptone (Fisher), 10 g/L yeast extract (Fisher). Flasks may be inoculated with yeast, such as 0.44 g/L of Ethanol Red or Red Star yeast (Fermentis) and incubated at 32° C. with agitation (about 200 rpm) at a pH of 4.5 with aeration (about 0.8 vvm for 100 L or 1.0 vvm for 5 L).

Another fermentation protocol used, referred to as F15MF9TP3, includes carrying out fermentation of 5 L or 100 L in a 14 L bioreactor containing culture medium with 250 g/L glucose, 20 g/L peptone (Fisher), 10 g/L yeast extract (Fisher). Flasks were inoculated with 0.44 g/L of Red Star yeast (Fermentis) and incubated at 32° C. with agitation (about 200 rpm) at a pH of 4.5 with aeration (<0.8 or 1 vvm) with an air LPM of 5.

A magnetic field may be applied to these protocols as described in more detail elsewhere herein.

To monitor cultures, samples from the medium may be removed at various time points to determine the glucose and ethanol concentrations, cell count, dissolved oxygen, optical density and BRIX using High Performance Liquid Chromatography (HPLC), YSI 2700 Biochemistry Analyzer (YSI Inc, Yellow Springs, Ohio), Cobas Mira Biochemistry Analyzer (F. Hoffmann-La Roche, Ltd, Nutley, N.J.), gas chromatography (Dombek et al (1986) Appl. Environ. Microbiol. 52:975 981) or other suitable techniques.

| | Absorbance | YSI | Cobas | HPLC | YSI | Cobas | HPLC | | | | |
|---|---|---|---|---|---|---|---|---|---|---|---|
| | Biomass | Glucose | Glucose | Glucose | Ethanol | Ethanol | Ethanol | Initial | | | |
| | dry wt max | max | max | max | max | max | max | pH | | Cobas | HPLC |
| | Growth | consumption | consumption | Consumption | Production | Production | Production | time | Final | Biomass | Biomass |
| | rate g/L/h | rate g/L/h | rate g/L/h | rate g/L/h | Rate g/L/h | Rate g/L/h | Rate g/L/h | used | pH | Yield | Yield |
| F9TP3 | 0.769 | −18 | −17.5 | −18.62 | 9.3 | 11.9 | 6.61 | | | 0.022 | 0.025 |
| F15MF9TP3 | 1.04 | −25.05 | −15.62 | −15.72 | 8.2 | 10.73 | 8.74 | | | 0.033 | 0.026 |

| | Absorbance | YSI | Cobas | HPLC | YSI | Cobas | HPLC | | | | |
|---|---|---|---|---|---|---|---|---|---|---|---|
| | Biomass | Glucose | Glucose | Glucose | Ethanol | Ethanol | Ethanol | | | | |
| | dry wt max | max | max | max | max | max | max | | | | |
| Overall | Growth | consumption | consumption | consumption | Production | Production | Production | Cobas | total | HPLC | YSI |
| 6 hr | rate g/L/h | rate g/L/h | rate g/L/h | rate g/L/h | Rate g/L/h | Rate g/L/h | Rate g/L/h | Yield | ferm | Yield | Yield |
| F9TP3 | 0.45 | −9.5 | −11.25 | −10.5 | 6.56 | 3.45 | | 4.28 | 0.3508 | 6.44 | |
| F15MF9TP3 | 0.87 | 12.51 | −12.78 | −14.4 | 7.4 | 5.82 | | 6.04 | 0.5173 | 0.44 | |

The Cobas data demonstrates that magnetic fermentation has a better growth rate, ethanol yield, and ethanol production rate.

| Test | What Tested |
|---|---|
| HPLC | Glucose & Ethanol |
| HPLC | Lactic Acid, Acetic Acid & Ethanol |
| Cobas Mira | Glucose & Ethanol |
| YSI 2700 | Glucose & Ethanol |
| BRIX | % Glucose |
| Dissolved | Percent oxygen in fermentor |
| Oxygen | |
| Cell Count | Yeast Cell Count |
| O.D. | Density of fluid |

TOTAL SAMPLES

| | |
|---|---|
| HPLC | 550 |
| YSI 2700 | 500 |
| COBAS MIRA | 800 |
| BRIX | 500 |
| TOTAL SAMPLES | 2350 |

### Example 2

### Dry Grind Corn Ethanol Process

Dry corn is ground, mixed with water producing a slurry, heat-treated through a jet cooker with alpha-amylase to swell the starch and break the starch into smaller polymers. This pasteurized corn mash is then fortified with urea as the nitrogen source, inoculated with active commercial yeast strain, and starch hydrolyzed to glucose by the addition of glucoamylase. The yeast converts one glucose molecule into two ethanol and two carbon dioxide molecules. Typically a 48 to 60 hour yeast fermentation will yield 18% ethanol by volume from a 32% corn mash by solids. The whole fermentation mash is then passed through a distillation column to remove the ethanol. This is followed by a low gravity centrifugation in which the solid portion called distillers grains and the supernatant called thin stillage which is then concentrated by flash evaporation into syrup are recovered. The syrup is added back onto the distiller grains, and dried to produce dried distiller grains with solubles (DDGS). The 95% ethanol recovered from the distiller is passed through a molecular sieve to remove the 5% water which produces fuel grade ethanol. The DDGS is ship to farmers around the world for animal feed, primarily for ruminant livestock.

In one exemplary embodiment, fermentation for use in ethanol production is described. The ethanol market is currently experiencing high growth. Ethanol is generally blended with gasoline at various levels to fuel motor vehicles. Due to limited supplies of crude oil and limitations in refining capacity, concerns over environmental degradation, and the resulting increase in gasoline prices, there appears to be a positive outlook for further growth in the ethanol market. Ethanol can be produced from various sources, including corn, barley, and wheat, as well as cellulose feedstocks. For purposes of this exemplary embodiment, corn is used to produce ethanol.

FIG. 22 illustrates one embodiment of a corn dry-milling process for ethanol production. As shown in FIG. 22, corn is provided and undergoes a corn cleaning process 304, followed by a hammermill process 306. In step 308 a slurry mixing step is performed and an enzyme, such as an alpha-amylase enzyme 309, is introduced. In step 310, liquefaction occurs, in step 316 magnetic fermentation takes place where carbon dioxide 318 is produced. In step 320, distillation occurs which produces ethyl alcohol 324. Whole stillage 322 is also produced. A centrifuge 326 is used to produce thin stillage 328, which may undergo additional cooking, returning to the cooker 312, or else the thin stillage is provided to an evaporator 330. The resulting coarse solids 332 are returned to a rotary dryer 334 and/or as a distillers wet grain 336 co-product. The solubles are provided as conditioned distillers soluble co-product 340. The rotary dryer 334 is also used to produce distillers dried grain with solubles 338. The process shown in FIG. 22 is merely one embodiment of a corn dry milling process. Each processor may have different or varying steps.

### Example 3

### Cellulose Bioconversion to Fuel Grade Ethanol

Cellulose is the most abundant organic compound in the biosphere and it is found in all plant materials as lignocellulose. Cellulose is plant cell wall (30-40%). Lignin is the cell cement that holds plant cells together (20-30%). Hemicellulose is found dispersed outside the plant cell (30-40%). Plant biomass (i.e., switch grass, corn stovers, wood chips, etc.) are dried in the field and stored in a reduced moisture environment. Bails of dried plant biomass is then ground, pretreated to remove microbial and enzymatic inhibitors, pasteurized at high temperatures, then fermented. The fermentation process includes the hydration (water contribution to the protein structure), as protein stability has been directly tied to the equilibrium of structuring water between low-density and higher density forms. It is believed that applying and controlling the magnetic field disrupts the water's hydrogen bonding, thus affecting the protein's structure by speeding-up its un-folding (denaturation).

The fermentation slurry contains pretreated ground plant biomass, cellulosic enzymes and yeast for ethanol bioconversions of glucose from cellulose and of possibly pentoses from hemicellulose. Residual co-products will have value in feed and non-feed applications (i.e., soil amendments, plastics, adhesives, asphalt, etc.).

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

Thus, methods and apparatus related to fermentation have been disclosed. The present invention contemplates numerous variation in the type of fermentation process, whether the magnets used are permanent or magnetic, the number and types of sensors used, the number and types of different controls, the methodology for controlling a DC power supply when used, and other variations. The present invention is not to be limited to this disclosure as these and other variations are contemplated which fall within the spirit and scope of the invention.

What is claimed is:

1. A method for magnetic fermentation, comprising:
subjecting a biological material in a medium to a magnetic field in order to affect fermentation of the biological material into a fermented product.

2. The method of claim 1 wherein the method further comprises determining whether the fermentation reaction occurs in an acidic or alkaline medium.

3. The method of claim 2 wherein the reaction occurs in an acidic medium, the method further comprising applying a positive magnetic field.

4. The method of claim 2 wherein the reaction occurs in an alkaline medium, the method further comprising applying a negative magnetic field.

5. The method of claim 1 wherein the biological material is a fermentable carbohydrate.

6. The method of claim 1 wherein the biological material is a sugar, biomass, or starch.

7. The method of claim 1 wherein the fermented product is ethanol.

8. The method of claim 1 wherein the fermented product is lactic acid.

9. The method of claim 1 wherein the method further comprises recovering the fermented product.

10. The method of claim 1 wherein the medium comprises a microorganism or an enzymatic preparation or a combination thereof for the conversion of the biological material to a fermented product.

11. The method of claim 10 wherein the microorganism is a yeast, bacteria, or fungus.

12. The method of claim 1 further comprising monitoring the magnetic field using a magnetic sensor.

13. The method of claim 1 further comprising monitoring the magnetic field using a submersible gauss meter submerged in the medium.

14. The method of claim 1 further comprising monitoring the fermentation with one or more sensors.

15. The method of claim 14 further comprising modulating the magnetic field based on information obtained during the step of monitoring the fermentation.

16. The method of claim 1 wherein the magnetic field is generated using permanent magnets.

17. The method of claim 1 wherein the magnetic field is generated using electromagnets.

18. The method of claim 1, wherein the magnetic field has a magnitude of between 2000 to 3000 Gauss.

19. The method of claim 1 wherein the magnetic field is a DC magnetic field.

20. The method of claim 1 wherein the magnetic field is a monopole magnetic field.

21. The method of claim 1 wherein the medium is water, a broth or a nutrient solution.

22. The method of claim 1 further comprising monitoring pH of the medium.

23. The method of claim 22 further comprising modulating the magnetic field to modulate the pH of the medium based on information obtained during the step of monitoring the pH of the medium.

24. The method of claim 1 further comprising determining the level of the biological material in the medium.

25. The method of claim 24 further comprising modulating the magnetic field based on information obtained during the step of determining the level of the biological material in the medium.

26. The method of claim 1 further comprising determining the level of the fermented product in the medium.

27. The method of claim 26 further comprising modulating the magnetic field based on information obtained during the step of determining the level of the fermented product in the medium.

28. The method of claim 1 further comprising monitoring the oxidation reduction potential of the medium.

29. The method of claim 28 further comprising modulating the magnetic field based on information obtained during the step of monitoring the oxidation reduction potential of the medium.

30. The method of claim 1 further comprising regulating the magnetic field at least partially based on pH of the medium.

31. The method of claim 1 wherein the effect on fermentation is increasing the rate of production of the fermented product.

32. The method of claim 1 wherein the effect on fermentation is increasing the yield of the fermented product.

33. The method of claim 1 wherein the effect on fermentation is shortening the length of time to produce a fermented product.

34. The method of claim 1 wherein the effect on fermentation is increasing the growth rate of the microorganism.

35. A method for producing ethanol, comprising:
subjecting to a monopole positive magnetic field of about 2000 to about 3000 Gauss a medium comprising a biological material and a microorganism or an enzymatic preparation or a combination thereof, wherein the microorganism or the enzymatic preparation is capable of fermenting the biological material to produce ethanol.

36. The method of claim 35 wherein the biological material is a fermentable carbohydrate.

37. The method of claim 35 further comprising preparing the biological material using a dry grind method.

38. The method of claim 35 further comprising preparing the biological material of biomass as a slurry.

39. The method of claim 35 wherein the ethanol is fuel grade ethanol.

40. The method of claim 35 wherein the biological material is a sugar, biomass, or starch.

41. The method of claim 35 wherein the step of subjecting a biological material and a microorganism in a medium to a positive magnetic field is performed using permanent magnets.

42. The method of claim 35 wherein the step of subjecting a biological material and a microorganism in a medium to a positive magnetic field is performed using electromagnets.

43. The method of claim 35 wherein the microorganism is a yeast, bacteria, or fungus or combinations thereof.

44. The method of claim 35 wherein the rate of ethanol production is increased as compared to a control that does not use a positive magnetic field.

45. The method of claim 35 wherein the yield of the fermented product is increased as compared to a control that does not use a positive magnetic field.

46. The method of claim 35 wherein the method further comprises recovering ethanol.

47. The method of claim 35 wherein the medium is water, a broth or a nutrient solution.

48. The method of claim 35 wherein the step of subjecting to a positive magnetic field is performed by applying the magnetic field to the medium within a conduit of a recycle stage.

49. The method of claim 48 wherein the step of applying the magnetic field is performed using a plurality of magnetic modules positioned around the conduit, each of the plurality of magnetic modules, each of the magnetic modules being substantially U-shaped for positioning on the conduit, each of the magnetic modules having a base and legs extending from the base with an electromagnet at the base and permanent magnets near the legs and wherein the plurality of magnetic modules being arranged in alternating fashion such that the base of each magnetic module is opposite the base of any immediately adjacent magnetic module.

50. The method of claim 48 wherein the step of applying the magnetic field uses a variable DC power supply electrically connected to the plurality of electromagnets.

51. A method for magnetic fermentation, comprising:
controlling a fermentation process by subjecting a biological material in a medium to a DC magnetic field having a magnitude of between 2000 to 3000 Gauss;
electronically monitoring the fermentation process to generate fermentation data;
wherein the step of controlling comprises adjusting the magnitude of the DC magnetic field.

52. A magnetic fermentation system, comprising:
a fermentation vessel for containing biological material and a medium;
a magnetic field component for applying a magnetic field to the medium during a fermentation process within the fermentation vessel;
wherein the magnetic field component is configured to create a magnetic field having a magnitude of between 2000 to 3000 Gauss.

53. The magnetic fermentation system of claim 52 wherein the magnetic field component comprises a permanent magnet.

54. The magnetic fermentation system of claim 52 wherein the magnetic field component comprises an electromagnet.

55. The magnetic fermentation system of claim 52 wherein the magnetic field component comprises a plurality of electromagnets.

56. The magnetic fermentation system of claim 52 further comprising an intelligent control electrically connected to the magnetic field component and adapted for controlling the magnetic field applied by the magnetic field component.

57. The magnetic fermentation system of claim 56 further comprising a magnetic field sensor adapted for sensing magnetic field of the medium and electrically connected to the intelligent control.

58. The magnetic fermentation system of claim 56 further comprising a sensor to measure pH associated with the vessel and electrically connected to the intelligent control.

59. The magnetic fermentation system of claim 52 wherein the magnetic field component is configured for applying the magnetic field to medium within staging operatively connected to the vessel.

60. The magnetic fermentation system of claim 59 wherein the staging comprises a recycle stage.

61. The magnetic fermentation system of claim 51 wherein the magnetic field component comprises a plurality of electromagnets electrically connected to a variable DC power supply.

62. A magnetic field generating device for applying a magnetic field to a fluid flowing through a conduit, comprising:
a plurality of magnetic modules, each of the magnetic modules being substantially U-shaped for positioning on the conduit, each of the magnetic modules having a base and legs extending from the base with an electromagnet at the base and permanent magnets near the legs;
wherein the plurality of magnetic modules being arranged in alternating fashion such that the base of each magnetic module is opposite the base of any immediately adjacent magnetic module.

63. The magnetic field generating device of claim 62 further comprising a variable DC power supply electrically connected to the plurality of magnetic modules.

64. The magnetic field generating device of claim 62 wherein the conduit forms a portion of a fermentation system.
